SEQUENCE LISTING (31 sequences)

SEQ ID NO: 1 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 21 aa, Homo sapiens
GIVEQCCTSICSLYQLENYCN

SEQ ID NO: 3 - protein, 30 aa, Homo sapiens
FVNQHLCGSHLVEALYLVCGERGFFYTPKT

SEQ ID NO: 4 - protein, 21 aa, Homo sapiens
  MISC_FEATURE (12)..(12): Xaa is Ala, Thr, Asp, Asn, Glu, Gln, His or Tyr
GIVEQCCTSICXLYQLENYCN

SEQ ID NO: 5 - protein, 21 aa, Homo sapiens
  MISC_FEATURE (13)..(13): Xaa is Ala, Glu, Gln, His, or Trp
GIVEQCCTSICSXYQLENYCN

SEQ ID NO: 6 - protein, 21 aa, Homo sapiens
  MISC_FEATURE (17)..(17): Xaa is Ala, Gln, His, Trp, or Tyr
GIVEQCCTSICSLYQLXNYCN

SEQ ID NO: 7 - protein, 21 aa, Homo sapiens
  MISC_FEATURE (12)..(12): Xaa is Ser, Ala, Thr, Asp, Asn, Glu, Gln, His or Tyr
GIVEQCCTSICXXYQLXNYCN

SEQ ID NO: 8 - protein, 21 aa, Homo sapiens
  MISC_FEATURE (8)..(8): Xaa is His, Glu, Gln, Arg, or Lys
GIVEQCCXSICXXYQLXNYCN

SEQ ID NO: 9 - protein, 30 aa, Homo sapiens
  MISC_FEATURE (13)..(13): Xaa is Ala, Asp, His, or Leu
FVNQHLCGSHLVXALYLVCGERGFFYTXXT

SEQ ID NO: 10 - protein, 30 aa, Homo sapiens
  MISC_FEATURE (17)..(17): Xaa is Glu, Gln, Ala, His, Trp, or Tyr
FVNQHLCGSHLVEALYXVCGERGFFYTXXT

SEQ ID NO: 11 - protein, 30 aa, Homo sapiens
  MISC_FEATURE (13)..(13): Xaa is Ala, Asp, His, or Leu
FVNQHLCGSHLVXALYXVCGERGFFYTXXT

SEQ ID NO: 12 - protein, 62 aa, Homo sapiens
  MISC_FEATURE (10)..(10): Xaa is Asp or His
FVNQHLCGSXLVXALYXVCGERGFFYTXXTGXXXXXXXXRRGIVEQCCXSICXXXQLXNYCN

SEQ ID NO: 13 - protein, 62 aa, Homo sapiens
  MISC_FEATURE (10)..(10): Xaa is  Asp or His
FVNQHLCGSXLVXALYXVCGERGFFYTXXTEEXXXXXXXRRGIVEQCCXSICXXXQLXNYCN

SEQ ID NO: 14 - protein, 62 aa, Homo sapiens
  MISC_FEATURE (10)..(10): Xaa is Asp or His
FVNQHLCGSXLVXALYXVCGERGFFYTXXTEXXXXXXXXSRGIVEQCCXSICXXXQLXNYCN

SEQ ID NO: 15 - protein, 21 aa, Homo sapiens
GIVEQCCTSICSEYQLENYCN

SEQ ID NO: 16 - protein, 30 aa, Homo sapiens
FVNQHLCGSHLVEALYLVCGERGFFYTKPT

SEQ ID NO: 17 - protein, 30 aa, Homo sapiens
  MISC_FEATURE (24)..(24): Xaa = para-chlorophenylalanine
FVNQHLCGSHLVEALYLVCGERGXFYTKPT

SEQ ID NO: 18 - protein, 21 aa, Homo sapiens
GIVEQCCTSICSHYQLENYCN

SEQ ID NO: 19 - protein, 21 aa, Homo sapiens
GIVEQCCTSICSHYQLENYCN

SEQ ID NO: 20 - protein, 21 aa, Homo sapiens
GIVEQCCTSICSWYQLENYCN

SEQ ID NO: 21 - protein, 30 aa, Homo sapiens
  MISC_FEATURE (24)..(24): Xaa = ortho-fluorophenylalanine
FVNQHLCGSDLVEALYLVCGERGXFYTKPT

SEQ ID NO: 22 - protein, 21 aa, Homo sapiens
GIVEQCCQSICSWYQLENYCN

SEQ ID NO: 23 - protein, 21 aa, Homo sapiens
GIVEQCCTSICSYYQLENYCN

SEQ ID NO: 24 - protein, 30 aa, Homo sapiens; identical to SEQ ID NO: 17
  MISC_FEATURE (24)..(24): Xaa = ortho-chlorophenylalanine

SEQ ID NO: 25 - protein, 30 aa, Homo sapiens; identical to SEQ ID NO: 17
  MISC_FEATURE (24)..(24): Xaa = cyclohexylalanine

SEQ ID NO: 26 - protein, 21 aa, Homo sapiens
GIVEQCCTSICSAYQLENYCN

SEQ ID NO: 27 - protein, 30 aa, Homo sapiens
FVNQHLCGSHLVEALYEVCGERGFFYTKPT

SEQ ID NO: 28 - protein, 21 aa, Homo sapiens
GIVEQCCTSICSFYQLENYCN

SEQ ID NO: 29 - protein, 21 aa, Homo sapiens
GIVEQCCTSICSLYQLENYCN

SEQ ID NO: 30 - protein, 21 aa, Homo sapiens
GIVEQCCQSICSWYQLENYCN

SEQ ID NO: 31 - protein, 30 aa, Homo sapiens
  MISC_FEATURE (24)..(24): Xaa = para-chlorophenylalanine
FVNQHLCGSHLVQALYLVCGERGXFYTKPT

(19) United States
(12) Patent Application Publication
(10) Pub. No.: US 2016/0083448 A1
(43) Pub. Date: Mar. 24, 2016

(54) SITE 2 INSULIN ANALOGUES

(71) Applicant: Michael A. WEISS, Moreland Hills, OH (US)

(72) Inventor: Michael A. Weiss, Moreland Hills, OH (US)

(73) Assignee: CASE WESTERN RESERVE UNIVERSITY, Cleveland, OH (US)

(21) Appl. No.: 14/774,109

(22) PCT Filed: Mar. 17, 2014
(86) PCT No.: PCT/US14/30387

§ 371 Date: Sep. 9, 2015

Related U.S. Application Data
(60) Provisional application No. 61/798,165, filed on Mar. 15, 2013.

(51) Int. Cl. C07K 14/62

(52) U.S. Cl. CPC C07K 14/62; A61K 38/00

(57) ABSTRACT

An insulin analogue contains one or more modifications at a distinct protein surface comprising one or more of the residues at position B13, B17, A12, A13, and/or A17. Formulations of the above analogues at successive strengths U-100 to U-1000 in soluble solutions a at least pH value in the range 6.8-8.0 either in the presence of zinc ions at a molar ratio of 2.2-10 zinc ions per six insulin analogue monomers or in the presence of fewer than 1 zinc ions per six insulin analogue monomers. Use of the above formulation in an insulin pump functionally integrated with a continuous glucose monitor and computer-based control algorithm as a closed-loop system. A method of treating a patient with diabetes mellitus comprises administering a physiologically effective amount of the insulin analogue to a patient by means of intravenous, intraperitoneal, or subcutaneous injection.

### STATEMENT REGARDING FEDERALLY SPONSORED RESEARCH OR DEVELOPMENT

This invention was made with government support under cooperative agreements awarded by the National Institutes of Health under grant numbers DK040949 and DK074176. The U.S. government may have certain rights to the invention.

### BACKGROUND OF THE INVENTION

This invention relates to polypeptide hormone analogues that exhibits enhanced pharmaceutical properties, such as altered pharmacokinetic and pharmacodynamic properties, i.e., conferring foreshortened duration of action relative to soluble formulations of the corresponding wild-type human hormone. More particularly, this invention relates to insulin analogues containing (i) one or more amino-acid substitutions in its “Site-2 receptor-binding surface” in conjunction optionally with (ii) one or more B-chain substitutions known in the art to accelerate the absorption of an insulin analogue from a subcutaneous depot into the blood stream. The insulins analogues of the present invention may optionally contain a connecting domain (C domain) between A- and B-chains (and so be described as single-chain analogues) and may optionally contain standard or non-standard amino-acid substitutions at other sites in the A- or B chains. The essential idea underlying the present invention is to enhance the safety and efficacy of rapid-acting analogues through the simultaneous incorporation of substitutions in the Site-2 receptor-binding surface of the hormone. This combination of substitutions confers “fast-on/fast-off” pharmacokinetic properties of utility in the prandial control of blood glucose concentration following subcutaneous injection as a method of treatment of diabetes mellitus and of further utility in the algorithm-based operation of closed-loop systems for the treatment of diabetes mellitus (“smart pumps”).

The engineering of proteins, including therapeutic agents and vaccines, may have broad medical and societal benefits. Naturally occurring proteins—as encoded in the genomes of human beings, other mammals, vertebrate organisms, invertebrate organisms, or eukaryotic cells in general—often contain two or more functional surfaces. A benefit of protein analogues would be to achieve selective modification of one or the other of these functional surfaces, such as to provide fine-tuning of biological activity. An example of a therapeutic protein is provided by insulin. The three-dimensional structure of wild-type insulin has been well characterized as a zinc hexamer, as a zinc-free dimer, and as an isolated monomer in solution (FIGS. 1 and 2). Wild-type human insulin and insulin molecules encoded in the genomes of other mammals bind to insulin receptors (IRs), each of which containing multiple domains and associated domain surfaces. The IR is a dimer of αβ half-receptors (designated (αβ)₂) wherein the α chain and β chain are the post-translational products of a single precursor polypeptide. The hormone-binding surfaces of the (αβ)₂ dimer has been classified as Site 1 and Site 2 in relation to the non-linear binding and kinetic properties of the receptor. This binding scheme is shown in schematic form in FIG. 3. Recent advances in structural and biochemical analysis of fragments of the IR ectodomain have shown that Site 1 consists of a trans-binding element formed by both a subunits in the (αβ)₂ dimer: the N-terminal L1 domain of one subunit and the C-terminal α-helix (αCT) of the other (Whittaker J, Whittaker I J, Roberts C T Jr, Phillips N B, Ismail-Beigi F, Lawrence M C, and Weiss M A. α-Helical element at the hormone-binding surface of the insulin receptor functions as a signaling element to activate its tyrosine kinase. Proc. Natl. Acad. Sci. USA 109, 1116-71 (2012)). The location of Site 2 is not well characterized but is proposed to comprise parts of the first and second fibronectin-homology domains.

The receptor-binding surfaces of insulin or insulin analogues may likewise be classified on a cognate basis: the respective Site-1-binding surface (classical receptor-binding surface) and Site 2-binding surface (non-classical receptor-binding surface). The Site-1-binding surface of insulin overlaps its dimer-forming interface in the B chain whereas the Site-2-binding surface overlaps its hexamer-forming interface. The Site 1 hormone-IR interface has recently been visualized at low resolution (Menting J G, Whittaker J, Margetts M B, Whittaker L J, Kong G K, Smith B J, Watson C J, Záková L, Kletvíková E, Jiráĉek J, Chan S J, Steiner D F, Dodson G G, Brzozowski A M, Weiss M A, Ward C W, and Lawrence M C. How insulin engages its primary binding site on the insulin receptor. Nature 493, 241-5 (2103)). Presumptive Site 2-related residues may be defined either based on kinetic effects of mutations or based on positions that are extrinsic to site 1 wherein mutations nonetheless impair binding. These criteria highlight the potential importance of non-classical residues A12, A13, A17, B13 and B17. Respective Site-1-related and Site-2-related surfaces are shown in relation to the surface of an insulin monomer in FIG. 4. Whereas substitutions known in the art to accelerate the absorption of insulin from a subcutaneous depot are ordinarily within and adjacent to the Site-1-binding surface of the hormone (such as at residues B24, B28 or B29), we envisaged that modification of the Site-2-binding surface could modulate the cellular duration of signaling by the hormone-receptor complex once engaged at the surface of a target cell or tissue. Although not wishing to be restricted by theory, we further envisioned that such foreshortening of the cellular duration of signaling would confer “fast-off” pharmacokinetic properties to a rapid-acting insulin analogue (“fast-on”) whose disassembly in the subcutaneous depot had been accelerated by substitutions within or adjacent to the Site-1-binding surface as known in the art (see FIG. 6 for rationale). Thus, such a novel combination of Site-1/Site-2-related substitutions would together confer the desirable combination of fast-on/fast-off pharmacokinetic properties of novel utility in the treatment of diabetes mellitus.

It is known in the art that modifications or substitutions within the classical receptor-binding surface of insulin may impair the in vitro affinity of the hormone for its receptor by up to ca. fivefold (e.g., from a dissociation constant of 0.05 nM to a dissociation constant of 0.25 nM) without significant effect on in vivo potency as assessment by the ability of the variant insulin, when injected subcutaneously or intravenously, to cause a reduction in blood glucose concentration. Such robustness is, at least in part, attributed to a compensating relationship between affinity and rate of clearance of the hormone from the bloodstream. Because binding to the IR mediates both insulin action and, to a large extent, insulin clearance, a reduction in affinity leads to a proportionate increase in the circulatory half-life and hence opportunity to effect biological signaling. Examples of such compensation have been disclosed in relation to insulin analogues in which the Phenylalanine at position B24 is substituted by Cyclohexanylalanine (Cha), disclosed in International Patent Application Ser. No. PCT/US12/46575 filed Jul. 13, 2012 and U.S. Prov. Pat. Application Ser. No. 61/755,020 filed Jan. 22, 2013 the disclosures of which are incorporated by reference herein. The non-planar aliphatic ring of Cha at position B24 (illustrated in FIG. 8) impairs receptor-binding affinity by ca. threefold but has no effect on the potency or pharmacodynamics properties of KP-insulin as tested in diabetic Sprague-Dawley rats (FIG. 9A). Additional examples have been provided by modified by fluoro-aromatic and chloro-aromatic substitutions at position B24 as illustrated in FIG. 7 (in relation to the wild-type dimer interface) and as disclosed in U.S. patent application Ser. Nos. 13/515,192, 13/018,011 filed Jul. 18, 2012 and Jan. 31, 2011 respectively, U.S. Provisional Patent Application No. 61/709,448 filed Oct. 4, 2012, International Patent Application Nos. PCT/US2010/60085 and PCT/US2012/62423 filed Dec. 13, 2010 and Oct. 29, 2012 respectively, which are incorporated by reference herein. Modification of Phe^{B24} in insulin Lispro ([Lys^{B28}, Pro^{B29}]-insulin, also designated KP-insulin (the active component of Humalog®); Eli Lilly and Co.) by a single chloro-substitution at the ortho position of the aromatic ring (2-Cl-Phe^{B24}) thus impairs the biochemical affinity of the variant hormone for the isolated IR in vitro by ca. threefold without change in its in vivo potency as assessed in Sprague-Dawley rats rendered diabetic by streptozotocin or as assessed in anesthetized non-diabetic adolescent Yorkshire pigs in which endogenous insulin secretion was suppressed by intravenous octreotide (data not shown). Even more dramatically, whereas a derivative of [Asp^{B10}, Lys^{B28}, Pro^{B29}]-insulin (DKP-insulin) containing 2-F-Phe^{B24} (ortho-monofluoro-phenylalalanine at position B24) exhibits a similar decrement in receptor-binding affinity (ca. 35(±5)% relative to KP-insulin), its potency and duration of signaling are enhanced in diabetic Sprague-Dawley rats (FIG. 9B) as disclosed in in U.S. patent application Ser. No. 13/018,011 filed Jan. 31, 2011 (which is incorporated by reference herein). It would be expected, therefore, that modifications or substitutions generally in the insulin molecule that introduce moderate perturbations to IR binding as evaluated in vitro would be well tolerated in vivo and be indistinguishable from wild-type insulin with respect to potency or (in the absence of effects on self-assembly) other pharmacological properties—or even more potent and prolonged as in the case of the 2-F-Phe^{B24}-DKP-insulin. It was therefore unexpected to discover that substitutions or modifications within Site 2 can markedly foreshorten the duration of insulin action in vivo (an alteration of pharmacodynamics) despite conferring perturbations to the biochemical affinity of the variant insulin for the IR of less than fivefold. Although side chains at positions B13, B17, A12, A13, and A17 are not thought to be engaged at the primary hormone-binding surface of the insulin receptor, alanine scanning mutagenesis has shown that single Alanine substitutions at Site-2-related positions affect relative receptor-binding affinities as follows: (position B13) 12(±3)%, (B17) 62(±14)%, (A12) 108(±28)%, (A13) 30(±7)%, and (A17) 56(±20)%.

Administration of insulin has long been established as a treatment for diabetes mellitus. A major goal of conventional insulin replacement therapy in patients with diabetes mellitus is tight control of the blood glucose concentration to prevent its excursion above or below the normal range characteristic of healthy human subjects. Excursions below the normal range are associated with immediate adrenergic or neuroglycopenic symptoms, which in severe episodes lead to convulsions, coma, and death. Excursions above the normal range are associated with increased long-term risk of microvascular disease, including retinapathy, blindness, and renal failure. Insulin is a small globular protein that plays a central role in metabolism in vertebrates. Insulin contains two chains, an A chain, containing 21 residues, and a B chain containing 30 residues; individual residues are indicated by the identity of the amino acid (typically using a standard three-letter code), the chain and sequence position (typically as a superscript). The hormone is stored in the pancreatic β-cell as a Zn²⁺-stabilized hexamer, but functions as a Zn²⁺-free monomer in the bloodstream. Insulin is the product of a single-chain precursor, proinsulin, in which a connecting region (35 residues) links the C-terminal residue of B chain (residue B30) to the N-terminal residue of the A chain. A variety of evidence indicates that it consists of an insulin-like core and disordered connecting peptide. Formation of three specific disulfide bridges (A6-A11, A7-B7, and A20-B19; labeled in FIG. 2) is thought to be coupled to oxidative folding of proinsulin in the rough endoplasmic reticulum (ER). Proinsulin assembles to form soluble Zn²⁺-coordinated hexamers shortly after export from ER to the Golgi apparatus. Endoproteolytic digestion and conversion to insulin occurs in immature secretory granules followed by morphological condensation. Crystalline arrays of zinc insulin hexamers within mature storage granules have been visualized by electron microscopy (EM).

There is a medical and societal need to engineer a rapid-acting two-chain insulin analogue or single-chain insulin analogue that combines (i) accelerated disassembly of an insulin complex in the subcutaneous depot with (ii) foreshortened duration of cellular signaling once the hormone-receptor complex is engaged at the surface of target cells or tissues. There is also an unmet need of a subset of patients treated with prandial insulin injections to avoid late post-prandial hypoglycemia and the unmet performance specifications of closed-loop algorithm-based pump systems (“smart pumps”) with respect to safety and efficacy. Feedback control in a smart pump would be made more robust by a foreshortened duration of signaling as effects of over-delivery events would be curtailed. It would be desirable, therefore, to provide a novel class of insulin analogues that combined modifications in the B chain designed to accelerate disassembly of an insulin complex with modifications elsewhere in the protein molecule that lead to foreshortened duration of signaling.

### SUMMARY OF THE INVENTION

It is, therefore, an aspect of the present invention to provide two-chain and single-chain insulin analogues that provide (i) rapid absorption into the blood stream due to substitutions or modifications in or adjoining the Site-1-related surface of the B chain and (ii) foreshortened duration of target cell signaling due to mutations or modifications of the Site-2-related surface of the A- and/or B chain. Site-2-related substitutions are modifications at one or more of the following positions: B13, B17, A12, A13, and A17. The analogues of the present invention contain at least a portion of the biological activity of wild-type insulin to direct a reduction in the blood glucose concentration on subcutaneous or intravenous injection. It is an aspect of the present invention that the isoelectric points of the analogues lie in the range 3.5-6.0 such that formulation as a clear soluble solution in the pH range 6.8-8.0 is feasible.

The analogues of the present invention may contain Histidine at position B10 and so be amenable to formulation as zinc insulin hexamers. Optionally, the analogues of the present invention may contain Aspartic Acid at position B10 when combined with a substitution or modification elsewhere in the protein such that the analogue exhibits an affinity for the IR is equal to or less than that of wild-type insulin (and so unlikely to exhibit prolonged residence times in the hormone-receptor complex) and an affinity for the Type 1 IGF-1 receptor is equal to or less than that of wild-type insulin (and so unlikely to exhibit IGF-I-related mitogenicity).

Pertinent to the present invention is the invention of novel foreshortened C domains of length 6-11 residues in place of the 36-residue wild-type C domain characteristic of human proinsulin. Single-chain insulin analogues provide a favorable approach toward the design of fibrillation-resistant insulin analogues amenable to formulation as zinc-free monomers. Such single-chain analogues may be designed to bear substitutions within or adjoining the Site-1-binding surface of the B chain such as to confer rapid-acting pharmacokinetics. Single-chain insulin analogues suitable to further modification at one or more positions selected from B13, B17, A12, A13, or A17 are as disclosed in U.S. patent application Ser. No. 12/989,399 (filed Oct. 22, 2010) and U.S. Pat. No. 8,192,957, which are incorporated by reference herein.

### BRIEF DESCRIPTION OF THE SEVERAL VIEWS OF THE DRAWINGS

FIG. 1. Representation of the structure of insulin in a typical pharmaceutical formulation and as an isolated monomer in the bloodstream. (A) The phenol-stabilized R₆ zinc hexamer. Axial zinc ions (overlaid) are shown as coincident black spheres coordinated by histidine side chains. The A-chain is shown in dark gray, and B-chain in medium gray (residues B1-B8) and light gray (B9-B30). (B) Structure of an insulin monomer. The A chain is shown in dark gray, and B chain in medium gray; disulfide brides are depicted as balls and sticks (labels are provided in FIG. 2).

FIG. 2. Representation of the structure of insulin dimer and core Beta-sheet. Residues B24-B28 (medium gray) for an anti-parallel Beta-sheet, repeated three times in the hexamer by symmetry. The A- and B chains are otherwise shown in light and dark gray, respectively. The position of Phe^{B24} is highlighted in the arrow in dark gray. Cystines are identified by sulfur atoms that are shown as spheres. Coordinates were obtained from T₆ hexamer (PDB 4INS).

FIG. 3. Model of insulin receptor: each cc subunit of the receptor contains two distinct insulin-binding sites: Site 1 (high affinity) and Site 2 (low affinity but critical to signal propagation). Specific insulin binding bridges the two cc subunits, in turn altering the orientation between β subunits, communicating a signal to the intracellular tyrosine kinase (TK) domain.

FIG. 4. Representation of the functional surfaces of insulin. Whereas the classical receptor-binding surface of insulin engages IR Site 1 (B 12, B16, B24-B26), its Site 2-related surface includes hexamer contacts Val^{B17} and Leu^{A13}; proposed Site 2 residues are shown (B13, B17, A12, A13, and A17) with addition of neighboring residue B10, which may contribute to both Sites 1 and 2. The A- and B chains are otherwise shown in light gray and dark gray, respectively.

FIG. 5. Position of Leu^{A13} on the surface of an insulin hexamer, dimer and monomer. Coordinates were obtained from R₆ hexamer (PDB 1TRZ).

FIG. 6. Rationale for the design and formulation of mealtime insulin analogues. Rapid dissociation of the zinc hexamer yields dimers and monomers able to enter the capillaries. Current mealtime insulin analogs contain standard substitutions at the edge of the core Beta-sheet.

FIG. 7. Structure-based design of para-Cl-Phe^{B24} modification. (A) Ribbon model of wild-type R₆ zinc insulin hexamer. The A- and B chains are shown in light and dark gray, the axial zinc ions (overlaid) as spheres, and Phe^{B24} side chains in medium gray. (B) Ribbon model of insulin dimer; the anti-parallel B24-B28 β-sheet is in middle. Coloring scheme as in panel A. (C) Stereo pair showing aromatic cluster within dimer interface: residues B24 and B24′, B25 and B25′, and B26 and B26′. (D) Predicted model of modified dimer interface; the para-chloro atoms at B24 (aromatic ring position 2) are shown as spheres (50% of van der Waals radii).

FIG. 8. Aromatic and non-aromatic ring systems. (A) Phe contains a planar six-carbon aromatic ring. (B) Cyclohexanylalanine (Cha) contains a non-planar six-carbon aliphatic ring. Three views of each amino acid are shown. In each panel ball-and-stick models are shown at top, and molecular surface models at bottom. Carbon and hydrogen atoms are medium and light gray, respectively, whereas oxygen (nitrogen) atoms are dark gray and nitrogen atoms are black.

FIG. 9. Pharmacodynamics of Insulin Analogues with Class-1-Related Modifications. Studies of glycemic response to subcutaneous injection of insulin Lispro (KP-insulin) or analogs in Sprague-Dawley rats rendered diabetic by streptozotocin. (A) Comparison of KP-insulin (solid diamonds) and Cha^{B24}-DKP-insulin (B24Cha KP, open squares) at a dose of 20 μg per rat. The relative affinity of Cha^{B24}-DKP-insulin is ca. 30(±5)%. (B) Comparison of KP-insulin (filled diamonds) and ortho-monofluoro-Phe^{B24}-DKP-insulin (2F B24 DKP, open squares) at a dose of 50 μg per rat. The relative affinity of 2-F-Phe^{B24}-DKP-insulin is ca. 35(±5)%.

FIG. 10. Receptor-Binding Studies. Competitive binding assays using immobilized lectin-purified isoform B of the human insulin receptor. (A) Comparison of Trp^{A13}-KP-insulin (triangles) and KP-insulin (squares). (B) Top panel, Comparison of Tyr^{A13}-KP-insulin (upright triangles) and wild-type human insulin (squares). Bottom panel, Comparison of 4-Cl-Phe^{B24} derivative of Trp^{A13}-KP-insulin (inverted triangles) and wild-type human insulin (squares). (The affinity of KP-insulin is similar to that of wild-type human insulin and so either provides a suitable control.)

FIG. 11. Pharmacodynamic Assay. (A) Comparison of blood glucose levels over time for Trp^{A13}-KP-insulin (triangles, A13W-KP) and KP-insulin (diamonds, KP) in relation to inactive control samples: diluent alone (circles) and an analogue containing a mutation in the Site-1-related surface that impairs receptor binding by ca. 100-fold (Trp^{A3}-KP-insulin; squares, A3W-KP). (B) Comparison of blood glucose levels over time for KP-insulin (filled diamonds) and Tyr^{A13}-KP-insulin (open circles, YA13-KP). (C) Comparison of 4-Cl-Phe^{B24}-Trp^{A13}-KP-insulin (squares, WA13 4Cl B24 KP), Trp^{A13}-KP-insulin (circles, WA13-KP), 2-Cl-Tyr^{A14}-KP-insulin (triangles, YA13 2CL B24 KP), and KP-insulin (diamonds); no diluent control was employed. In panel A the fall in blood glucose concentration (as a percentage relative to the mean initial value; ca. 400 mg/dl) is shown as a function of time following subcutaneous injection whereas in panels B and C the mean glucose concentrations are shown. The extent of beginning hyperglycemia was similar in each set of rats on each date. Doses were in each case 60 μg per rat. Symbols are also defined as inset at bottom right of each panel.

FIG. 12. Circular Dichroism Spectra. Far-ultraviolet CD spectra of insulin analogues containing substitutions at position A13 are shown in relation to the CD spectrum of the parent analog KP-insulin. The legend is shown at upper right and includes the following: KP-insulin, (YA13 B24 Cha KP) Cha^{B24}-Tyr^{A13}-insulin, (WA13 B24 4Cl KP) 4-Cl-Phe^{B24}-Trp^{A13}-KP-insulin, (WA13 KP) Trp^{A13}-KP-insulin, (YA13 B24 4Cl KP) 4-Cl-Phe^{B24}-Tyr^{A13}-KP-insulin, and (YA13 KP), Tyr^{A13}-KP-insulin. The insulin analogues were made ca. 60 μM in 50 mM potassium phosphate (pH 7.4) at a temperature of 25° C.

FIG. 13. Chemical Denaturation Studies. CD-detected studies of protein unfolding as a function of the concentration of denaturant guanidine hydrochloride (horizontal axis). Symbols are defined in legend: (solid black squares, KP) KP-insulin, (circles, YA13 Cha KP) Cha^{B24}-Tyr^{A13}-KP-insulin, (triangles) 4-Cl-Phe^{B24}-Trp^{A13}-KP-insulin, (inverted triangles) Trp^{A13}-KP-insulin, (diamonds) 4-Cl-Phe^{B24}-Tyr^{A13}-KP-insulin, and (rotated triangles), Tyr^{A13}-KP-insulin. Ellipticity was monitored at a wavelength of 222 nm.

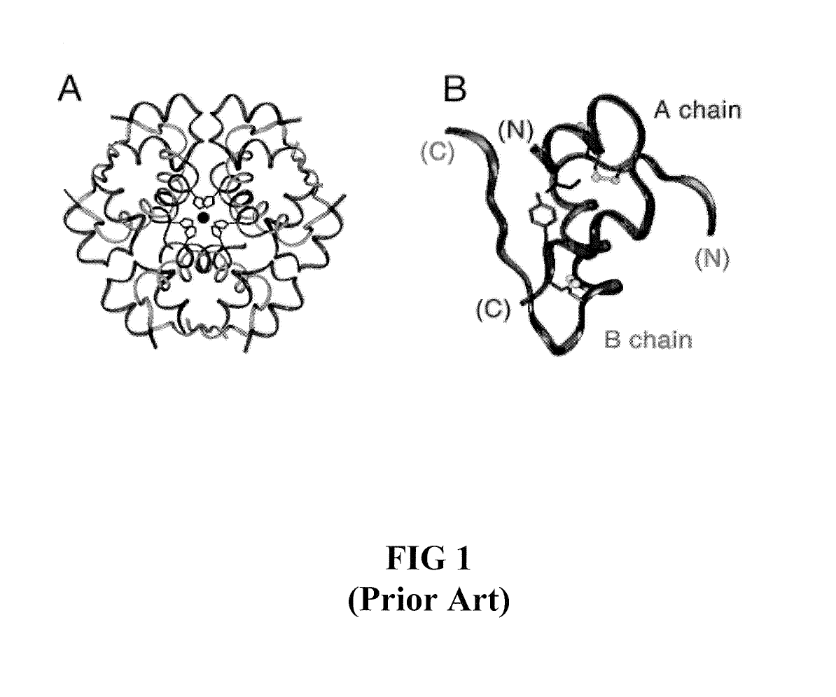

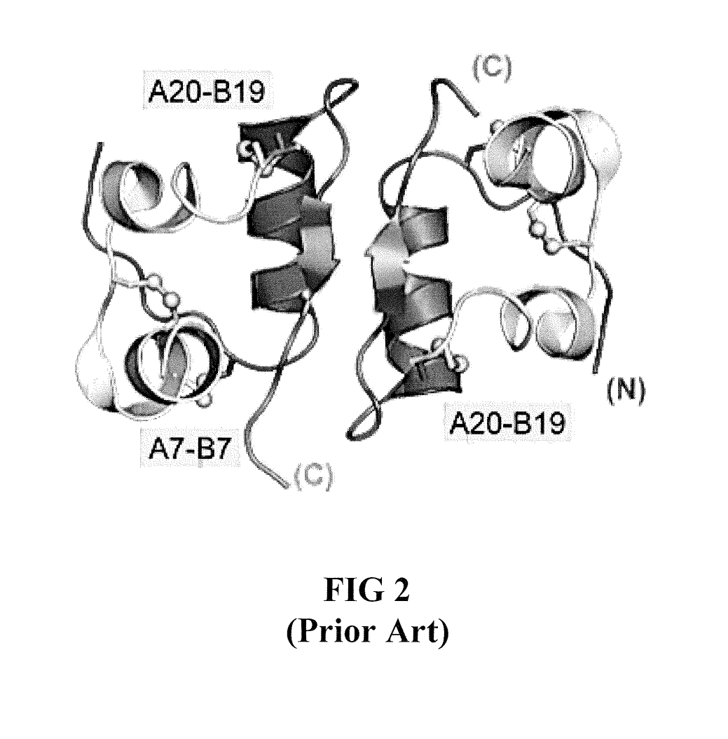

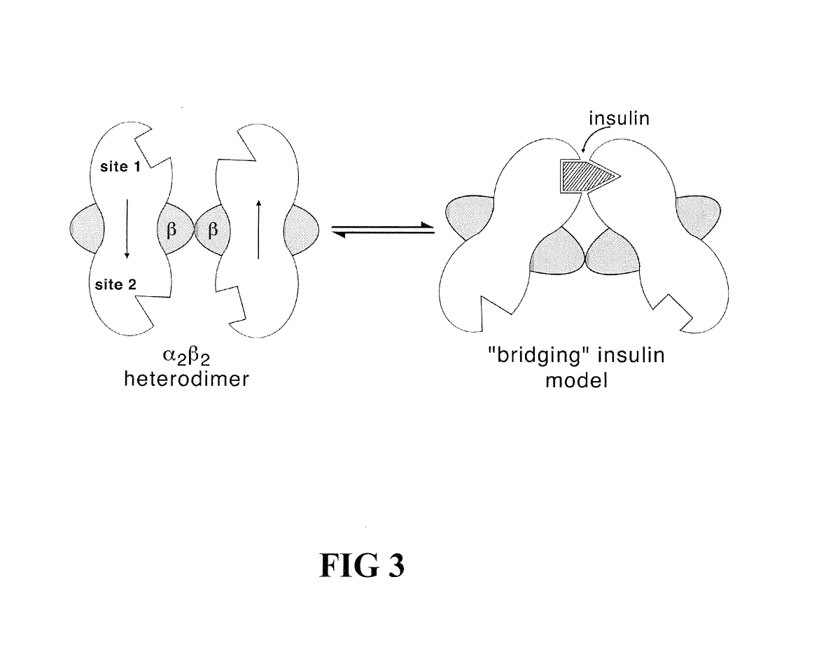

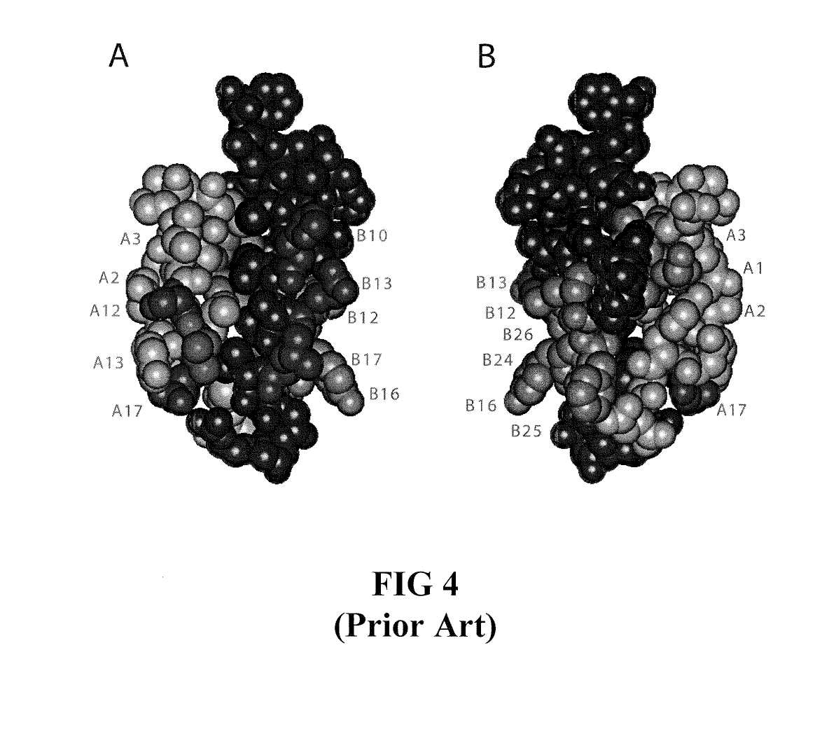

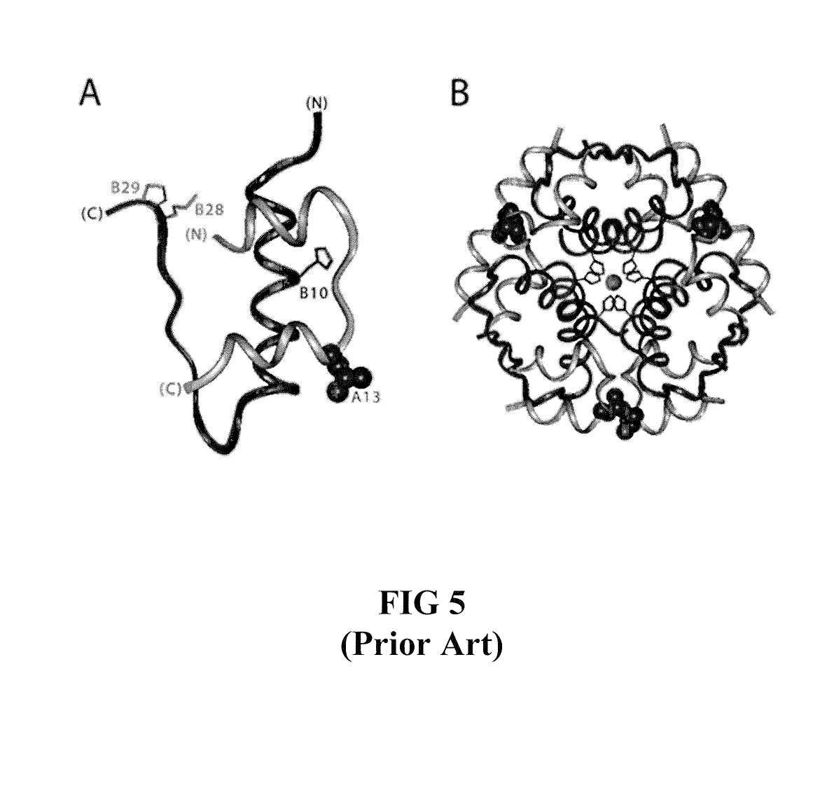

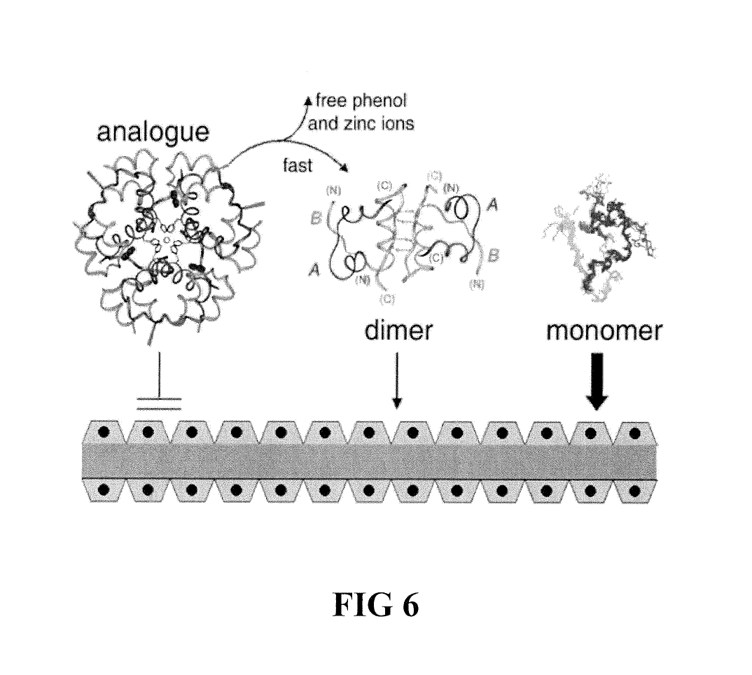

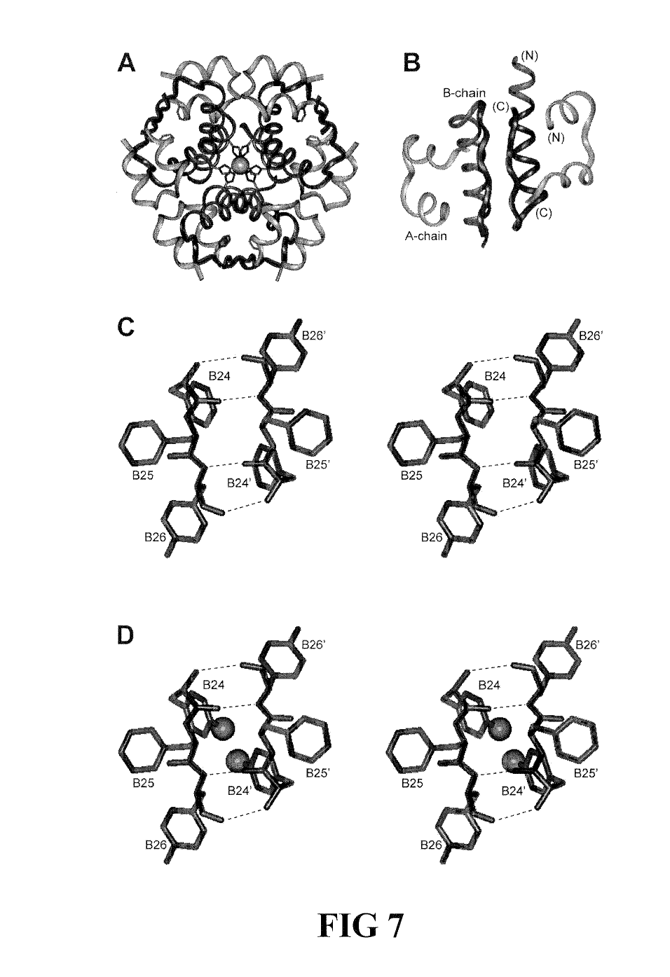

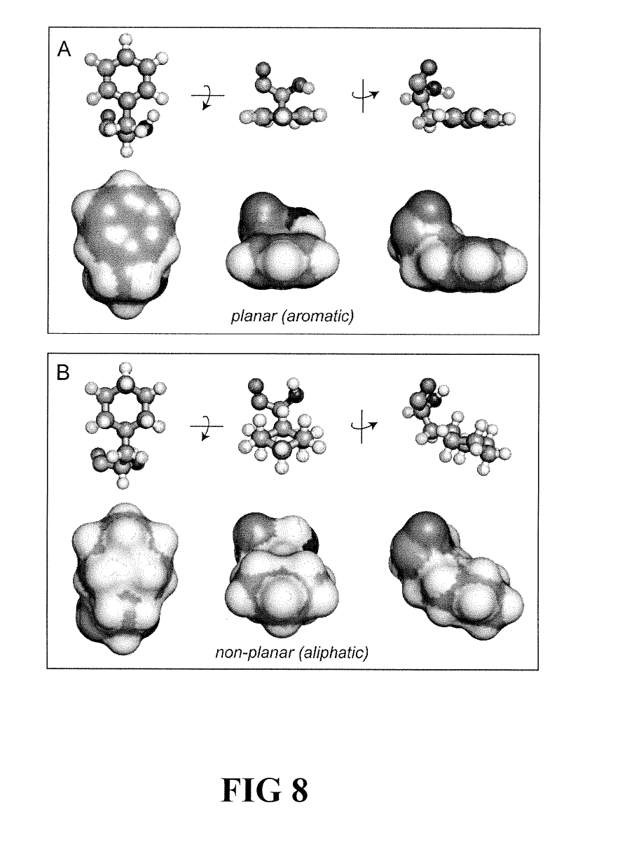

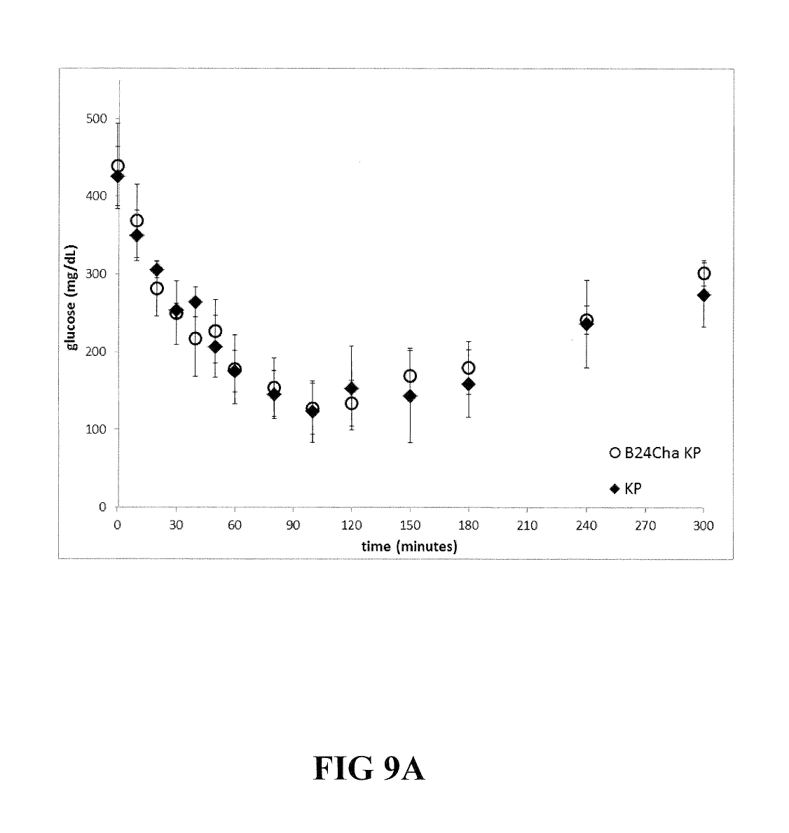

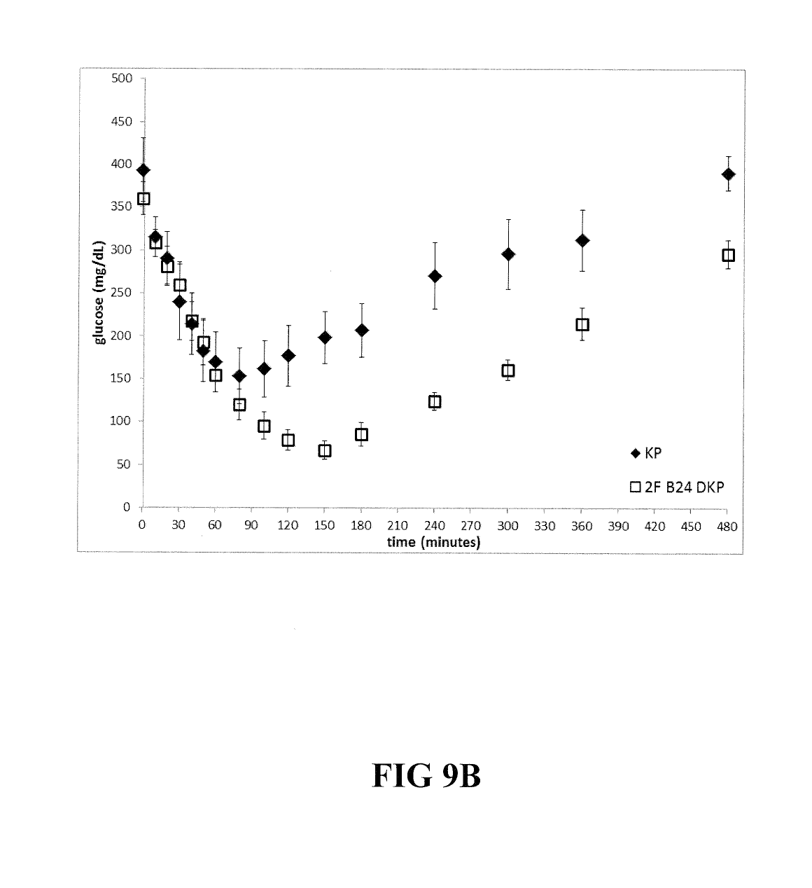

### DETAILED DESCRIPTION OF THE INVENTION

The present invention is directed toward a two-chain or single-chain insulin analogue that provides both (i) rapid absorption from a subcutaneous depot and (ii) foreshortened duration of action, a ratio of IR-A/IR-B receptor-binding affinities similar to that of wild-type insulin with absolute affinities in the range 5-100% (the lower limit chosen to correspond to proinsulin). Examples of B-chain substitutions to confer rapid absorption are Aspartic Acid or Lysine at position B28, optionally combined with Proline at position B29. Removal of Proline from position B28 is associated with decreased strength of dimerization and hexamer assembly irrespective of the nature of the substituted amino acid. Yet another example of B-chain substitutions that confer rapid absorption is the combination of Lysine at position B3 and Glutamic Acid at position B29 when formulated in the absence of zinc ions. Amino-acid substitutions introduced to effect foreshortened duration of signaling may be at one or more of the following positions: B13, B17, A12, A13, and A17. Examples of such substitutions are provided by (but not restricted to) Tryptophan, Tyrosine (except at A13), Alanine, Histidine, Glutamic Acid (except at B13 and A17), and Glutamine (except at B13). It is a feature of the present invention that the isoelectric point of the single-chain analogue is between 3.5 and 6.0 such that a soluble formulation neutral conditions (pH 6.8-8.0) would be feasible.

It is also envisioned that single-chain analogues may also be made with A- and B-domain sequences derived from animal insulins, such as porcine, bovine, equine, and canine insulins, by way of non-limiting examples. In addition or in the alternative, the insulin analogue of the present invention may contain a deletion of residues B1-B3 or may be combined with a variant B chain lacking Lysine (e.g., Lys^{B29} in wild-type human insulin) to avoid Lys-directed proteolysis of a precursor polypeptide in yeast biosynthesis in Pichia pastoris, Saccharomyces cerevisciae, or other yeast expression species or strains. The B-domain of the single-chain insulin of the present invention may optionally contain non-standard substitutions, such as D-amino-acids at positions B20 and/or B23 (intended to augment thermodynamic stability, receptor-binding affinity, and resistance to fibrillation), a halogen modification at the 2 ring position of Phe^{B24} (i.e., ortho-F-Phe^{B24}, ortho-Cl-Phe^{B24}, or ortho-Br-Phe^{B24}; intended to enhance thermodynamic stability and resistance to fibrillation), 2-methyl ring modification of Phe^{B24} (intended to enhance receptor-binding affinity). It is also envisioned that Thr^{B27}, Thr^{B30}, or one or more Serine residues in the C-domain may be modified, singly or in combination, by a monosaccaride adduct; examples are provided by O-linked N-acetyl-β-D-galactopyranoside (designated GalNAc-O^{β}-Ser or GalNAc-O^{β}-Thr), O-linked α-D-mannopyranoside (mannose-O^{β}-Ser or mannose-O^{β}-Thr), and/or α-D-glucopyranoside (glucose-O^{β}-Ser or glucose-O^{β}-Thr).

Furthermore, in view of the similarity between human and animal insulins, and use in the past of animal insulins in human patients with diabetes mellitus, it is also envisioned that other minor modifications in the sequence of insulin may be introduced, especially those substitutions considered “conservative.” For example, additional substitutions of amino acids may be made within groups of amino acids with similar side chains, without departing from the present invention. These include the neutral hydrophobic amino acids: Alanine (Ala or A), Valine (Val or V), Leucine (Leu or L), Isoleucine (Ile or I), Proline (Pro or P), Tryptophan (Trp or W), Phenylalanine (Phe or F) and Methionine (Met or M). Likewise, the neutral polar amino acids may be substituted for each other within their group of Glycine (Gly or G), Serine(Ser or S), Threonine (Thr or T), Tyrosine (Tyr or Y), Cysteine (Cys or C), Glutamine (Glu or Q), and Asparagine (Asn or N). Basic amino acids are considered to include Lysine (Lys or K), Arginine (Arg or R) and Histidine (His or H). Acidic amino acids are Aspartic acid (Asp or D) and Glutamic acid (Glu or E). Unless noted otherwise or wherever obvious from the context, the amino acids noted herein should be considered to be L-amino acids. Standard amino acids may also be substituted by non-standard amino acids belong to the same chemical class. By way of non-limiting example, the basic side chain Lys may be replaced by basic amino acids of shorter side-chain length (Ornithine, Diaminobutyric acid, or Diaminopropionic acid). Lys may also be replaced by the neutral aliphatic isostere Norleucine (Nle), which may in turn be substituted by analogues containing shorter aliphatic side chains (Aminobutyric acid or Aminopropionic acid).

The amino-acid sequence of human proinsulin is provided, for comparative purposes, as SEQ ID NO: 1.

(human proinsulin)
SEQ ID NO: 1
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Ala-Glu-Asp-
Leu-Gln-Val-Gly-Gln-Val-Glu-Leu-Gly-Gly-Gly-Pro-
Gly-Ala-Gly-Ser-Leu-Gln-Pro-Leu-Ala-Leu-Glu-Gly-
Ser-Leu-Gln-Lys-Arg-Gly-Ile-Val-Glu-Gln-Cys-Cys-
Thr-Ser-Ile-Cys-Ser-Leu-Tyr-Gln-Leu-Glu-Asn-Tyr-
Cys-Asn

The amino-acid sequence of the A chain of human insulin is provided as SEQ ID NO: 2.

(human A chain)
SEQ ID NO: 2
Gly-Ile-Val-Glu-Gln-Cys-Cys-Thr-Ser-Ile-Cys-Ser-
Leu-Tyr-Gln-Leu-Glu-Asn-Tyr-Cys-Asn

The amino-acid sequence of the B chain of human insulin is provided as SEQ ID NO: 3.

(human B chain)
SEQ ID NO: 3
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr

The amino-acid sequence of the A chain of human insulin modified at position A12 is provided as SEQ ID NO: 4.

(variant human A chain)
SEQ ID NO: 4
Gly-Ile-Val-Glu-Gln-Cys-Cys-Thr-Ser-Ile-Cys-Xaa-
Leu-Tyr-Gln-Leu-Glu-Asn-Tyr-Cys-Asn

Where Xaa indicates Ala, Thr, Asp, Asn Glu, Gln, His or Tyr.

The amino-acid sequence of the A chain of human insulin modified at position A13 is provided as SEQ ID NO: 5.

(variant human A chain)
SEQ ID NO: 5
Gly-Ile-Val-Glu-Gln-Cys-Cys-Thr-Ser-Ile-Cys-Ser-
Xaa-Tyr-Gln-Leu-Glu-Asn-Tyr-Cys-Asn

Where Xaa indicates Ala, Glu, Gln, His, Tyr or Trp.

The amino-acid sequence of the A chain of human insulin modified at position A17 is provided as SEQ ID NO: 6.

(variant human A chain)
SEQ ID NO: 6
Gly-Ile-Val-Glu-Gln-Cys-Cys-Thr-Ser-Ile-Cys-Ser-
Leu-Tyr-Gln-Leu-Xaa-Asn-Tyr-Cys-Asn

Where Xaa indicates Ala, Gln, His, Trp, or Tyr.

The amino-acid sequence of the A chain of human insulin modified at one or more of the positions A12, A13, and/or A17 is provided as SEQ ID NO: 7.

(variant human A chain)
SEQ ID NO: 7
Gly-Ile-Val-Glu-Gln-Cys-Cys-Thr-Ser-Ile-Cys-Xaa₁-
Xaa₂-Tyr-Gln-Leu-Xaa₃-Asn-Tyr-Cys-Asn

Where at least one of the Xaa sites contains a substitution relative to wild-type human insulin and wherein Xaa₁ indicates Ser, Ala, Thr, Asp, Asn Glu, Gln, His or Tyr; where Xaa₂ indicates Leu, Ala, Glu, Gln, His, or Trp; and where Xaa₃ indicates Glu, Ala, Gln, His, Trp, or Tyr.

The amino-acid sequence of the A chain of human insulin modified at residue A8 and also modified at one or more of the positions A12, A13, and/or A17 is provided as SEQ ID NO: 8.

(variant human A chain)
SEQ ID NO: 8
Gly-Ile-Val-Glu-Gln-Cys-Cys-Xaa₁-Ser-Ile-Cys-Xaa₂-
Xaa₃-Tyr-Gln-Leu-Xaa₄-Asn-Tyr-Cys-Asn

Where at least one of the Site-2-related sites (A12, A13, and A17) contains a substitution relative to wild-type human insulin and wherein Xaa₂ indicates Ser, Ala, Thr, Asp, Asn Glu, Gln, His or Tyr; where Xaa₃ indicates Leu, Ala, Glu, Gln, His, or Trp; and where Xaa₄ indicates Glu, Ala, Gln, His, Trp, or Tyr; and where Xaa₁ indicates His, Glu, Gln, Arg, or Lys.

The amino-acid sequence of a variant B chain of human insulin modified at position B13 is provided as SEQ ID NO: 9.

(variant human B chain)
SEQ ID NO: 9
Phe-Val-Asn-Gln-His-Leu-Cys-Gly-Ser-His-Leu-Val-
Xaa₃-Ala-Leu-Tyr-Leu-Val-Cys-Gly-Glu-Arg-Gly-Phe-
Phe-Tyr-Thr-Xaa₁-Xaa₂-Thr

Where Xaa₃ indicates Ala, Asp, His, or Leu; where Xaa₁ indicates any amino acid excluding Glycine, Tryptophan, Phenylalanine, Tyrosine, and Cysteine; and where Xaa₂ indicates Pro, Glu or Lys.

The amino-acid sequence of the B chain of human insulin modified at position B 17 is provided as SEQ ID NO: 10.

(variant human B chain)
SEQ ID NO: 10
Phe-Val-Asn-Gln-His-Leu-Cys-Gly-Ser-His-Leu-Val-
Glu-Ala-Leu-Tyr-Xaa₃-Val-Cys-Gly-Glu-Arg-Gly-
Phe-Phe-Tyr-Thr-Xaa₁-Xaa₂-Thr

Where Xaa₃ indicates Glu, Gln, Ala, His, Trp, or Tyr; where Xaa₁ indicates any amino acid excluding Glycine, Tryptophan, Phenylalanine, Tyrosine, and Cysteine; and where Xaa₂ indictes Pro, Glu, or Lys.

The amino-acid sequence of a variant B chain of human insulin modified at both positions B13 and B17 is provided as SEQ ID NO: 11.

(variant human B chain)
SEQ ID NO: 11
Phe-Val-Asn-Gln-His-Leu-Cys-Gly-Ser-His-Leu-Val-
Xaa₁-Ala-Leu-Tyr-Xaa₂-Val-Cys-Gly-Glu-Arg-Gly-
Phe-Phe-Tyr-Thr-Xaa₃-Xaa4-Thr

Where Xaa₁ indicates Ala, Asp, His, or Leu; where Xaa₂ indicates Gln, Glu, Ala, His, Trp, or Tyr; where Xaa₃ indicates any amino acid excluding Glycine, Tryptophan, Phenylalanine, Tyrosine, and Cysteine; and where Xaa₄ indictes Pro, Glu, or Lys.

Amino-acid sequences of single-chain insulin analogues of the present invention are given in SEQ ID NO 12-14.

SEQ ID NO: 12
Phe-Val-Asn-Gln-His-Leu-Cys-Gly-Ser-Xaa₁-Leu-Val-
Xaa₁-Ala-Leu-Tyr-Xaa₂-Val-Cys-Gly-Glu-Arg-Gly-
Phe-Phe-Tyr-Thr-Xaa₆-Xaa₇-Thr-Gly-Z-Arg-Arg-Gly-
Ile-Val-Glu-Gln-Cys-Cys-Xaa₈-Ser-Ile-Cys-Xaa₃-
Xaa₄-Xaa₉-Gln-Leu-Xaa₅-Asn-Tyr-Cys-Asn

Where at least one of the Site-2-related sites (B13, B17, A12, A13, and A17) contains a substitution relative to wild-type human insulin wherein Xaa₁ indicates Glu, Ala, Asp, His, or Leu; where Xaa₂ indicates Leu, Glu, Gln, Ala, His, Trp, or Tyr; where Xaa₃ indicates Ser, Ala, Thr, Asp, Asn, Glu, Gln, Tyr, or His; where Xaa₄ indicates Leu, Ala, Glu, Gln, His, Tyr,or Trp; where Xaa₅ indicates Glu, Gln, Ala, His, Trp, Tyr or Leu; where Xaa₁ indictes His or Asp; where Xaa₆ indicates any amino acid excluding Glycine, Tryptophan, Phenylalanine, Tyrosine, and Cysteine; where Xaa₇ indictes Pro or Lys; Xaa₈ indictes Glu, Gln, His, Arg, Lys or Ornithine; where Xaa₉ indicates Tyr or Glu; and where Z indicates a polypeptide segment of length 3-8.

SEQ ID NO: 13
Phe-Val-Asn-Gln-His-Leu-Cys-Gly-Ser-Xaa₁-Leu-Val-
Xaa₁-Ala-Leu-Tyr-Xaa₂-Val-Cys-Gly-Glu-Arg-Gly-
Phe-Phe-Tyr-Thr-Xaa₆-Xaa₇-Thr-Glu-Glu-Z-Arg-Arg-
Gly-Ile-Val-Glu-Gln-Cys-Cys-Xaa₈-Ser-Ile-Cys-
Xaa₃-Xaa₄-Xaa₉-Gln-Leu-Xaa₅-Asn-Tyr-Cys-Asn

Where at least one of the Site-2-related sites (B13, B17, A12, A13, and A17) contains a substitution relative to wild-type human insulin wherein Xaa₁ indicates Glu, Ala, Asp, His, or Leu; where Xaa₂ indicates Leu, Glu, Gln, Ala, His, Trp, or Tyr; where Xaa₃ indicates Ser, Ala, Thr, Asp, Asn, Glu, Gln, Tyr, or His; where Xaa₄ indicates Leu, Ala, Glu, Gln, His, Tyr, or Trp; where Xaa₅ indicates Glu, Gln, Ala, His, Trp, Tyr or Leu; where Xaa₁ indictes His or Asp; where Xaa₆ indicates any amino acid excluding Glycine, Tryptophan, Phenylalanine, Tyrosine, and Cysteine; where Xaa₇ indictes Pro or Lys; Xaa₈ indictes Glu, Gln, His, Arg, Lys or Ornithine; where Xaa₉ indicates Tyr or Glu; and where Z indicates a polypeptide segment of length 2-7.

SEQ ID NO: 14
Phe-Val-Asn-Gln-His-Leu-Cys-Gly-Ser-Xaa₁-Leu-Val-
Xaa₁-Ala-Leu-Tyr-Xaa₂-Val-Cys-Gly-Glu-Arg-Gly-
Phe-Phe-Tyr-Thr-Xaa₆-Xaa₇-Thr-Glu--Z-Ser-Arg-Gly-
Ile-Val-Glu-Gln-Cys-Cys-Xaa₈-Ser-Ile-Cys-Xaa₃-
Xaa₄-Xaa₉-Gln-Leu-Xaa₅-Asn-Tyr-Cys-Asn

Where at least one of the Site-2-related sites (B13, B17, A12, A13, and A17) contains a substitution relative to wild-type human insulin wherein Xaa₁ indicates Glu, Ala, Asp, His, or Leu; where Xaa₂ indicates Leu, Glu, Gln, Ala, His, Trp, or Tyr; where Xaa₃ indicates Ser, Ala, Thr, Asp, Asn, Glu, Gln, Tyr, or His; where Xaa₄ indicates Leu, Ala, Glu, Gln, His, Tyr,or Trp; where Xaa₅ indicates Glu, Gln, Ala, His, Trp, Tyr or Leu; where Xaa₁ indictes His or Asp; where Xaa₆ indicates any amino acid excluding Glycine, Tryptophan, Phenylalanine, Tyrosine, and Cysteine; where Xaa₇ indictes Pro or Lys; Xaa₈ indictes Glu, Gln, His, Arg, Lys or Ornithine; where Xaa₉ indicates Tyr or Glu; and where Z indicates a polypeptide segment of length 3-8.

Analogous synthetic genes have been prepared in a subset of cases and cloned in Pichia pastoris. For production of two-chain insulin analogues, a 53-residue mini-proinsulin precursor was expressed, folded, and secreted by P. pastoris by means of an N-terminal signal peptide essentially as described (Kjeldsen T, Pettersson A F, Hach M. The role of leaders in intracellular transport and secretion of the insulin precursor in the yeast Saccharomyces cerevisiae. J. Biotechnol. 75, 195-208 (1999)). The codon encoding position A13 was altered by site-directed mutagenesis to encode Trp, Tyr, His, or Glu. Trp^{A13} and Tyr^{A13} analogues (SEQ. ID. NO:5) were selected for initial characterization.

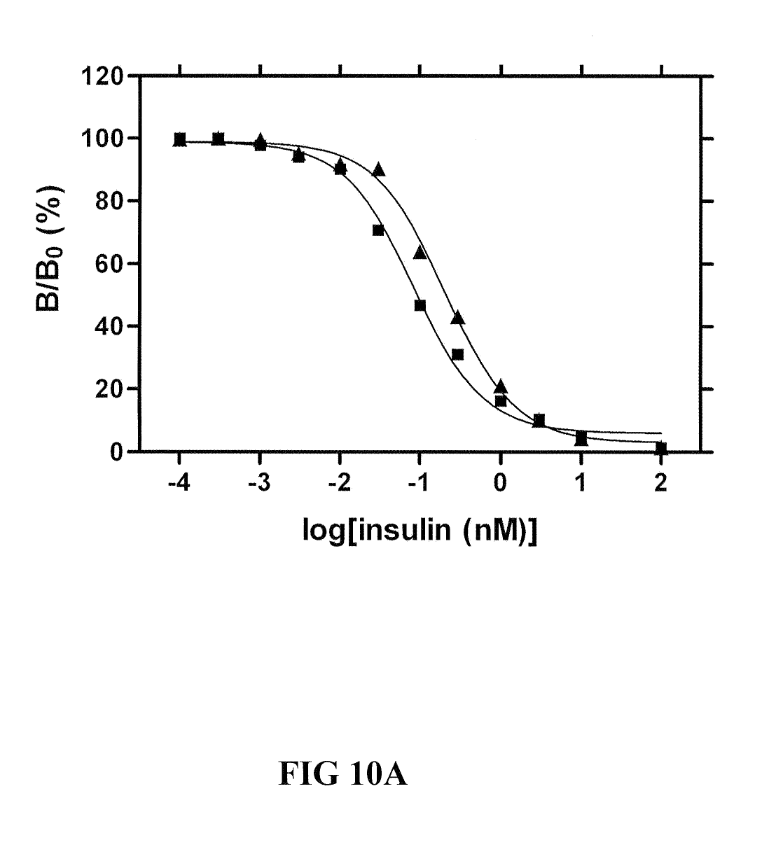

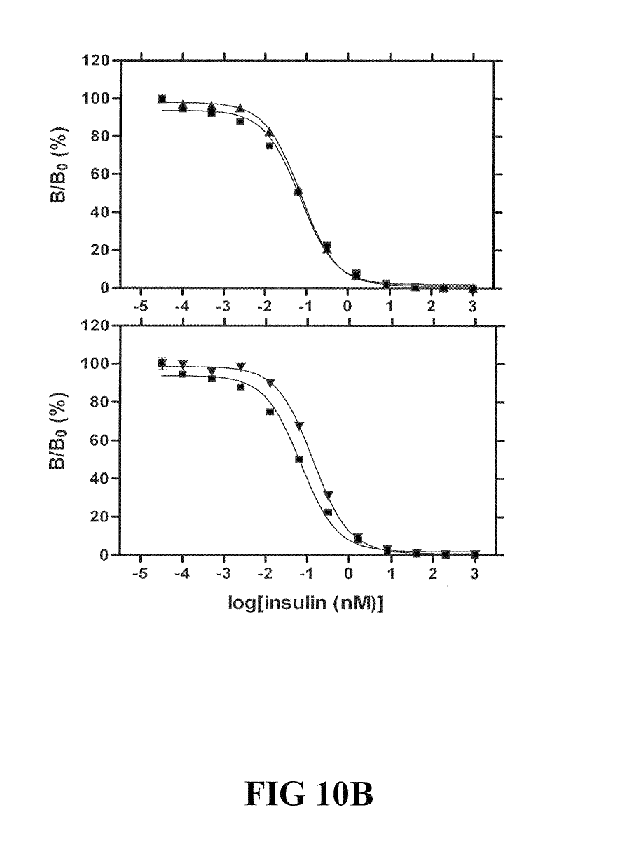

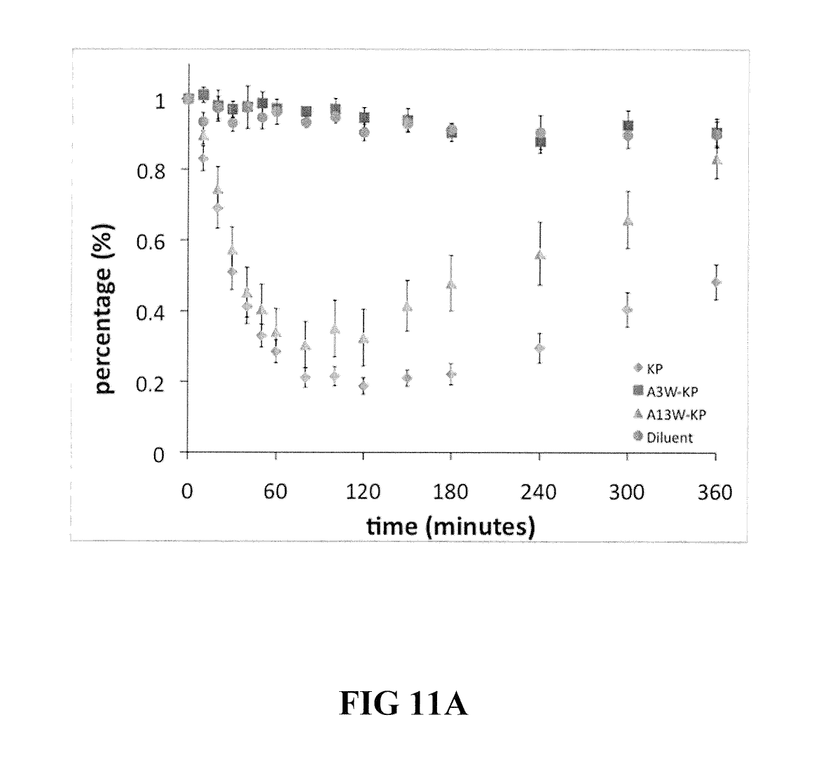

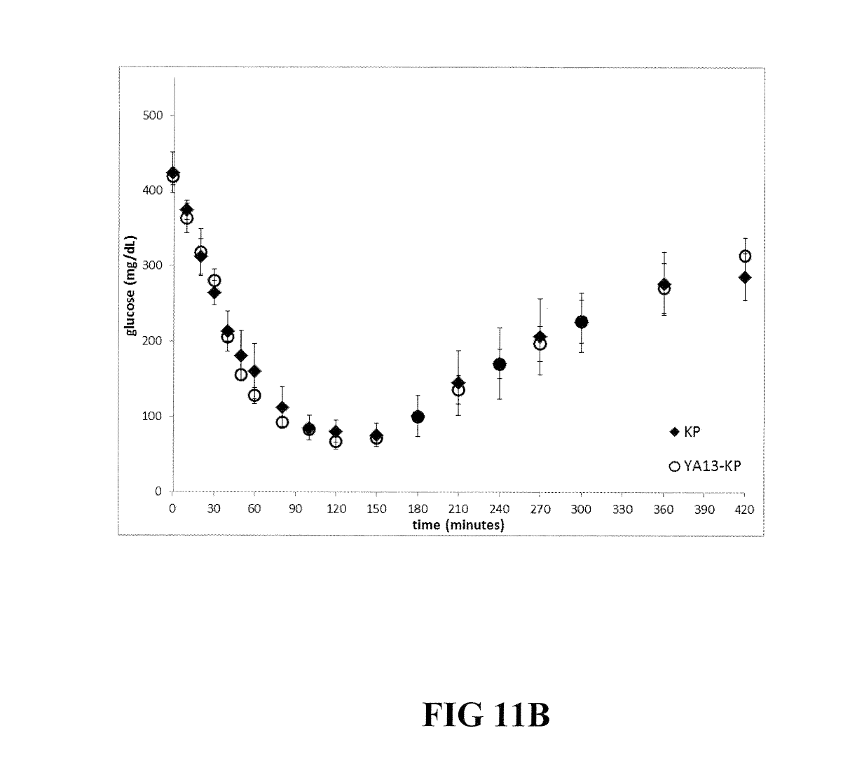

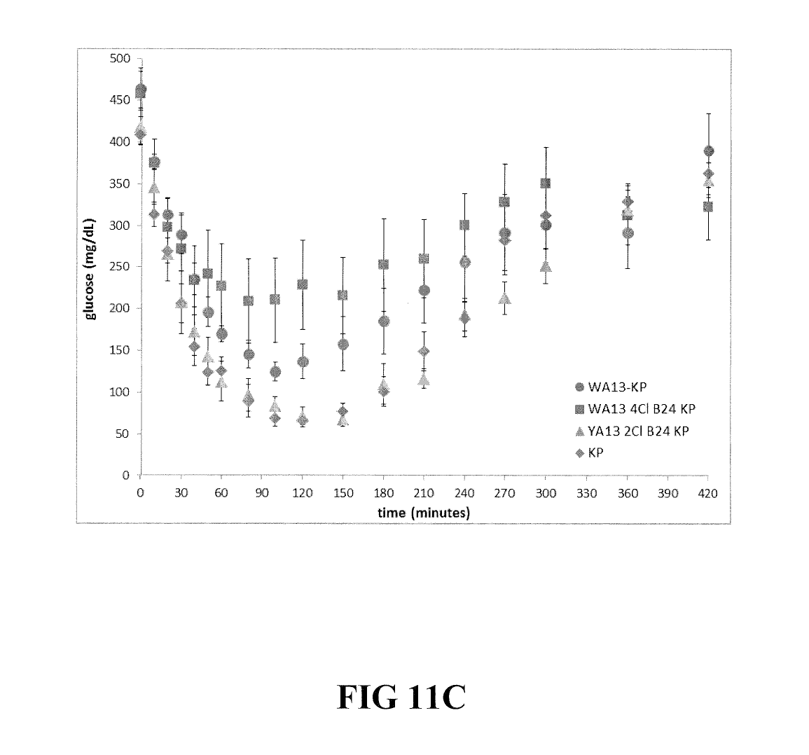

We observed that Trp^{A13} (SEQ ID NO: 20) in Site 2 impairs binding of KP-insulin to the IR by ca. twofold (Table 1 and FIG. 10A) whereas the effect of Tyr^{A13} (SEQ ID NO: 23) on binding affinity is negligible (Table 1 and FIG. 10B, top). Thus, comparison of Trp^{A13}-KP-insulin to control analogue Tyr^{A13}-KP-insulin provided a test of the guiding hypothesis that modest perturbations to biochemical affinity by a Site-2 modification would lead to foreshortened duration of insulin action in vivo; i.e., the long-sought but unmet goal of a “fast-off” pharmacodynamics property. We therefore undertook to investigate the pharmacodynamics properties and relative potencies of these and related insulin analogues in a rat model of diabetes mellitus as described in detail below. These results were obtained as shown in FIGS. 10 and 11. The foreshortening of the pharmacodynamic (PD) properties of Trp^{A13}-KP-insulin was further enhanced by co-modification with 4-Cl-Phe^{B24} (FIG. 11C; SEQ ID. NO: 17). Although this modification has negligible effect on the receptor-binding affinity (FIG. 10B, bottom relative to parent TrpA13-KP-insulin in FIG. 10A), synergistic improvement is observed in the extent of foreshortening. Such synergy between modifications at the Site-1-related surface and Site-2-related surface is desirable from the perspective of prandial insulin therapy and the robust and safe operation of closed-loop systems. The shorter the duration of action, the more quickly a closed-loop system can compensate for over-delivery events.

Analogs of KP-insulin containing Trp^{A13} or Tyr^{A13} were prepared by trypsin-catalyzed semi-synthesis. The essential idea was to use trypsin “in reverse” as a synthetic enzyme in an organic co-solvent. This protocol employs (i) a synthetic octapeptide representing residues (N)-GXFYTKPT “KP” substitutions (underlined) and (ii) truncated analog des-octapeptide[B23-B30]-insulin (DOI), Trp^{A13}-DOI, or Tyr^{A13}-DOI. Because the octapeptide differs from the wild-type B23-B30 sequence (GFFYTPKT), protection of the lysine ε-amino group is not required during trypsin treatment. The protocol was extended to enable co-modification of the A13 site with unnatural amino-acid substitutions at position B24 as contained in the synthetic octapeptide. Wild-type DOI was prepared from human or porcine insulin; the A13 analogues of DOI were generated by trypsin digestion of a 53-residue mini-proinsulin (MPI) precursor modified at the A13 codon as expressed and secreted by yeast strain Pichia pastoris. In each case the three native disulfide bridges are retained throughout the procedure.

In brief, des-octapeptide insulin (150 mg) and octapeptide (150 mg) were dissolved in a mixture of dimethylacetamide/1,4-butandiol/0.2 M Tris acetate (pH 8) containing 10 mM calcium acetate and 1 mM ethylene diamine tetra-acetic acid (EDTA) (35:35:30, v/v, 4 ml). The 5-fold molar excess of octapeptide over DOI ensured that the reverse reaction of trypsin (proteolysis) was prevented by substrate saturation. The final pH was adjusted to 7.0 with 0.1 ml of N-methylmorpholine. The solution was cooled to 12° C., and 15 mg of TPCK-trypsin were added and incubated for 2 days at 12° C. An additional 15 mg of tryp sin were added after 24 hr. The reaction was then be acidified with 0.1% trifluoroacetic acid and purified by preparative reverse-phase HPLC (C4). The product was verified by mass spectrometry (MALDI-TOF-TOF; Applied Biosystems, Foster City, Calif.). KP-octapeptides containing Phe^{B24}, 2-Cl-Phe^{B24}, 4-Cl-Phe^{B24}, or Cha^{B24} (each ca. 600 mg crude material) were provided by the CCF Peptide Core Facility.

DOI and DOI analogues were generated by trypsin digestion of human insulin, available in bulk quantity from insulin manufacturers. To generate DOI or DOI analogue, the insulin (300 mg) was added to a solution of 0.1 M ammonium bicarbonate (60 ml) containing IM urea. Trypsin (30 mg) was first dissolved in 1.0 ml of distilled deionized water and then added to the protein solution; cleavage proceeds for 48 hours. DOI or DOI analogue was purified from trypsin, unreacted insulin, and any other contaminants by preparative reverse-phase HPLC using a C4 column. Yields of at least 150 mg purified DOI were typically obtained. Analytical reverse-phase HPLC (C18) was used to follow the time course of semi-synthesis and assess the purity of polypeptide reagents and products by analytical HPLC and MALDI-TOF MS (below) for semi-quantitative estimation of products <1%.

Receptor-binding affinities for the Trp^{A13} and Tyr^{A13} derivatives of KP-insulin were determined by an in vivo competitive displacement assay as illustrated in FIG. 10. The protocol for assay of receptor-binding activities was as follows. Microtiter strip plates (Nunc Maxisorb) were incubated overnight at 4° C. with AU5 IgG (100 μl/well of 40 mg/ml in phosphate-buffered saline). Binding data were analyzed by a two-site sequential model. Data were corrected for nonspecific binding (amount of radioactivity remaining membrane associated in the presence of 1 M human insulin. In all assays the percentage of tracer bound in the absence of competing ligand was less than 15% to avoid ligand-depletion artifacts. Dissociation constants (K_{d}) were determined by fitting to a mathematic model as described by Whittaker and Whittaker (2005. J. Biol. Chem. 280, 20932-20936); the model employed non-linear regression with the assumption of heterologous competition (Wang, 1995, FEBS Lett. 360, 111-114). Results are summarized in Table 1. Whereas Tyr^{A13} does not affect receptor binding of KP-insulin, Trp^{A13} results in a reduction of binding affinity by ca. twofold. Modest changes were also observed with co-substitution of Cha^{B24}, 2-Cl-Phe^{B24}, or 4-Cl-Phe^{B24} in accordance with previously disclosed studies.

TABLE 1
Properties of Insulin Analogues

| | receptor-binding | thermodynamic |
|---|---|---|
| protein | affinity | stability (25° C.) |
| wt human insulin | 100% | 3.3(±0.1) kcal/mole |
| KP-insulin | 90-100 | 2.9(±0.1) kcal/mole |
| Trp^{A13}-KP-insulin | 50-60 | 2.6(±0.1) kcal/mole |
| 4-Cl-Phe^{B24}-Trp^{A13}-KP-ins. | 45-55 | 2.5(±0.1) kcal/mole |
| Tyr^{A13}-KP-insulin | 90-110 | 2.8(±0.1) kcal/mole |
| 4-Cl-Phe^{B24}-Tyr^{A13}-KP-ins. | ND | 2.5(±0.1) kcal/mole |
| Cha^{B24}-Trp^{A13}-KP-ins. | ND | 2.1(±0.1) kcal/mole |

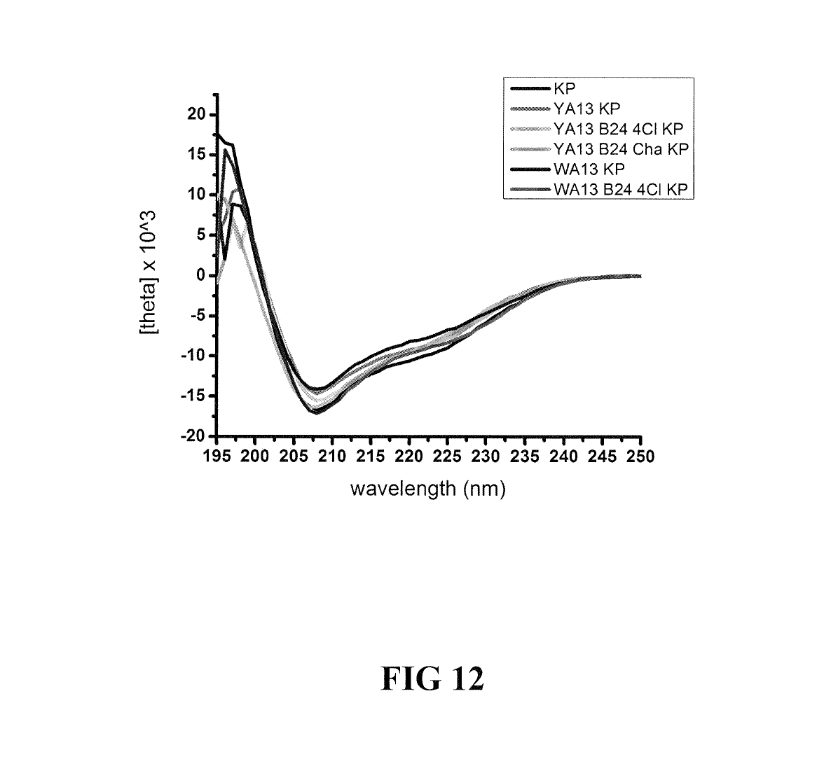

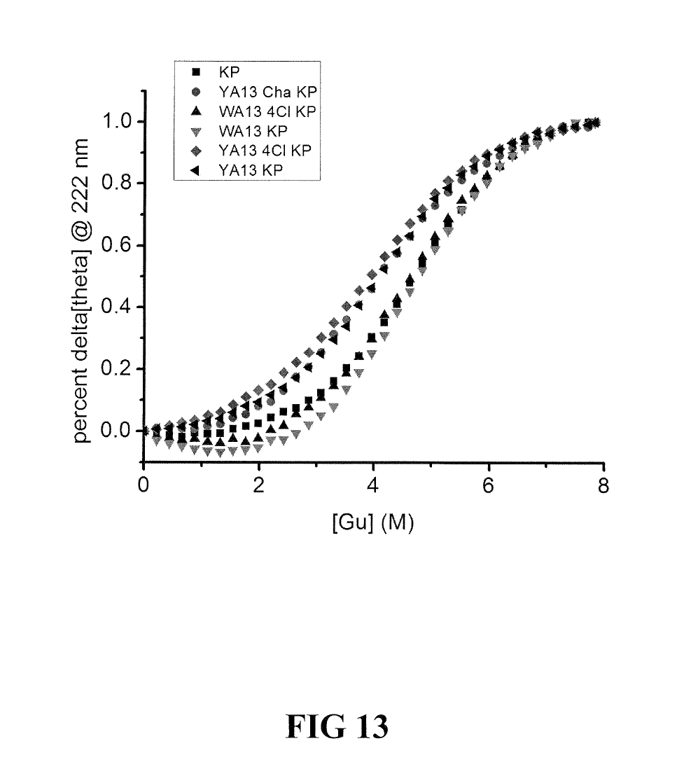

Circular dichroism (CD) spectra were obtained at 25° C. using an Aviv spectropolarimeter (Weiss et al., Biochemistry 39, 15429-15440) as shown in FIG. 12. The CD pattern is in each case consistent with a predominance of alpha-helix; variations are observed that may reflect small perturbations in the stability of secondary structure or may represent superimposed CD bands arising from the additional or modified aromatic side chains. Samples contained ca. 60 μM KP-insulin or analogues in 50 mM potassium phosphate (pH 7.4); samples were diluted to 5 μM for guanidine-induced denaturation studies at 25° C. Representative guanidine titrations are shown in FIG. 13. To extract free energies of unfolding, denaturation transitions were fitted by non-linear least squares to a two-state model as described by Sosnick et al., Methods Enzymol. 317, 393-409. In brief, CD data θ(x), where x indicates the concentration of denaturant, were fitted by a nonlinear least-squares program according to

$$θ ( x ) = \frac{{θ}_{A} + {θ}_{B} {e}^{( - Δ {G}_{{H}_{2} O}^{o} - mx ) / RT}}{1 + {e}^{- ( Δ {G}_{{H}_{2} O}^{o} mx ) / RT}}$$

where x is the concentration of guanidine and where θ_{A} and θ_{B} are baseline values in the native and unfolded states. Baselines were approximated by pre- and post-transition lines θ_{A}(x)=θ_{A}^{H}^{₂}^{O}+m_{A}x and θ_{B}(x)=θ_{B}^{H}^{₂}^{O}+m_{B}x. The m values obtained in fitting the variant unfolding transitions are lower than the m value obtained in fitting the wild-type unfolding curve. Estimates of free energies of unfolding, as obtained from application of this two-state model, are provided in Table 1.

To evaluate the biological activity and potency of the analogues in an animal model, male Sprague-Dawley rats (mean body mass ˜300 grams) were rendered diabetic by treatment with streptozotocin (STZ). Protein solutions containing KP-insulin (insulin Lispro, the active component of Humalog®), wild-type human insulin, and/or a two-chain or single-chain insulin of the present invention. A control was provided by injection of protein-free Lilly diluent (obtained from Eli Lilly and Co.) composed of 16 mg glycerin, 1.6 mg meta-cresol, 0.65 mg phenol, and 3.8 mg sodium phosphate pH 7.4. The activity of the insulin analogues was evaluated in relation to that of Humulog® (U-100 strength taken from an unexpired commercial vial). 20 or 60 micrograms of each of these formulations were injected subcutaneously, and resulting changes in blood glucose concentration were monitored by serial measurements using a clinical glucometer (Hypoguard Advance Micro-Draw meter). Rats were injected subcutaneously at time t=0 in groups of five (N=4-6). Blood was obtained from the clipped tip of the tail at time 0 and every 10 minutes up to 360 min. Representative two-chain analogues of the present invention, Trp^{A14}-KP-insulin of the present invention were found, under conditions of formulation similar to that of Humalog®, to retain a substantial proportion of the biological activity of insulin and with duration of action foreshortened with respect to Humalog®. Representative pharmacodynamic data are shown in FIG. 11. Various analogues according to the claimed invention are provided in Table 2.

TABLE 2

| Analogue | Sequences |
|---|---|
| GluA13 LysB28 | A-chain GIVEQCCTSICSEYQLENYCN (SEQ ID NO: 15) |
| ProB29 | B-chain FVNQHLCGSHLVEALYLVCGERGFFYTKPT |
| | (SEQ ID NO: 16) |
| GluA13 | A-chain GIVEQCCTSICSEYQLENYCN (SEQ ID NO: 15) |
| parachloro- | B-chain FVNQHLCGSHLVEALYLVCGERGXFYTKPT, |
| PheB24 LysB28 | X = parachlorophenylalanine (SEQ ID NO: 17) |
| ProB29 | |
| HisA13 LysB28 | A-chain GIVEQCCTSICSHYQLENYCN (SEQ ID NO: 18) |
| ProB29 | B-chain FVNQHLCGSHLVEALYLVCGERGFFYTKPT |
| | (SEQ ID NO: 16) |
| HisA13 | A-chain GIVEQCCTSICSHYQLENYCN (SEQ ID NO: 19) |
| parachloro- | B-chain FVNQHLCGSHLVEALYLVCGERGXFYTKPT, |
| PheB24 LysB28 | X = parachlorophenylalanine (SEQ ID NO: 17) |
| ProB29 | |
| TrpA13 LysB28 | A-chain GIVEQCCTSICSWYQLENYCN (SEQ ID NO: 20) |
| ProB29 | B-chain FVNQHLCGSHLVEALYLVCGERGFFYTKPT |
| | (SEQ ID NO: 16) |
| TrpA13 | A-chain GIVEQCCTSICSWYQLENYCN (SEQ ID NO: 20) |
| parachloro- | B-chain FVNQHLCGSHLVEALYLVCGERGXFYTKPT, |
| PheB24 LysB28 | X = parachlorophenylalanine (SEQ ID NO: 17) |
| ProB29 | |
| TrpA13 AspB10 | A-chain GIVEQCCTSICSWYQLENYCN (SEQ ID NO: 20) |
| orthofluoro- | B-chain FVNQHLCGSDLVEALYLVCGERGXFYTKPT, |
| PheB24 | X = orthofluorophenylalanine (SEQ ID NO: 21) |
| LysB28 ProB29 | |
| GlnA8 TrpA13 | A-chain GIVEQCCQSICSWYQLENYCN (SEQ ID NO: 22) |
| LysB28 ProB29 | B-chain FVNQHLCGSHLVEALYLVCGERGFFYTKPT |
| | (SEQ ID NO: 16) |
| GlnA8 TrpA13 | A-chain GIVEQCCQSICSWYQLENYCN (SEQ ID NO: 22) |
| parachloro- | B-chain FVNQHLCGSHLVEALYLVCGERGXFYTKPT, |
| PheB24 LysB28 | X = parachlorophenylalanine (SEQ ID NO: 17) |
| ProB29 | |
| TyrA13 LysB28 | A-chain GIVEQCCTSICSYYQLENYCN (SEQ ID NO: 23) |
| ProB29 | B-chain FVNQHLCGSHLVEALYLVCGERGFFYTKPT |
| | (SEQ ID NO: 16) |
| TyrA13 | A-chain GIVEQCCTSICSYYQLENYCN (SEQ ID NO: 23) |
| parachloro- | B-chain FVNQHLCGSHLVEALYLVCGERGXFYTKPT, |
| PheB24 LysB28 | X = parachlorophenylalanine (SEQ ID NO: 17) |
| ProB29 | |
| TyrA13 | A-chain GIVEQCCTSICSYYQLENYCN (SEQ ID NO: 23) |
| orthochloro- | B-chain FVNQHLCGSHLVEALYLVCGERGXFYTKPT, |
| PheB24 LysB28 | X = orthochlorophenylalanine (SEQ ID NO: 24) |
| ProB29 | |
| TyrA13 | A-chain GIVEQCCTSICSYYQLENYCN (SEQ ID NO: 23) |
| cyclohexylala- | B-chain FVNQHLCGSHLVEALYLVCGERGXFYTKPT, |
| nine-B24 | X = cyclohexylalanine (SEQ ID NO: 25) |
| LysB28 ProB29 | |
| AlaA13 LysB28 | A-chain GIVEQCCTSICSAYQLENYCN (SEQ ID NO: 26) |
| ProB29 | B-chain FVNQHLCGSHLVEALYLVCGERGFFYTKPT |
| | (SEQ ID NO: 16) |
| GluB17 LysB28 | A-chain GIVEQCCTSICSLYQLENYCN (SEQ ID NO: 2) |
| ProB29 | B-chain FVNQHLCGSHLVEALYEVCGERGFFYTKPT |
| | (SEQ ID NO: 27) |
| PheA13 LysB28 | A-chain GIVEQCCTSICSFYQLENYCN (SEQ ID NO: 28) |
| ProB29 | B-chain FVNQHLCGSHLVEALYLVCGERGFFYTKPT |
| | (SEQ ID NO: 16) |
| PheB17 LysB28 | A-chain GIVEQCCTSICSLYQLENYCN (SEQ ID NO: 29) |
| ProB29 | B-chain FVNQHLCGSHLVEALYFVCGERGFFYTKPT |
| | (SEQ ID NO: 16) |
| GlnA8 TrpA13 | A-chain GIVEQCCQSICSWYQLENYCN (SEQ ID NO: 30) |
| GlnB13 | B-chain FVNQHLCGSHLVQALYLVCGERGXFYTKPT, |
| parachloro- | X = parachlorophenylalanine (SEQ ID NO: 31) |
| PheB24 LysB28 | |
| ProB29 | |

Receptor binding by the various analogues of the claimed invention was analyzed as follows. In vitro activity assays employed epitope-tagged holoreceptor of either human insulin receptor isoform B (hIR-B) and/or isoform A (hIR-A) and/or the homologous human type 1 insulin-like growth factor receptor (hIGFR) immobilized on 96 well plates. Relative activity is defined as the ratio of specific dissociation constants as determined by competitive displacement of bound ¹²⁵I-TyrA14 human insulin (in the case of IR) or ¹²⁵I-Tyr31 human IGF-I (in the case of IGFR). Dissociation constants (K_{d}) were determined by fitting to a mathematic model as described by Whittaker and Whittaker (2005. J. Biol. Chem. 280, 20932-20936); the model employed non-linear regression with the assumption of heterologous competition (Wang, 1995, FEBS Lett. 360, 111-114). Results listed in Table 3 (Assay: hIR-A, hIR-B) are consistent with native in vivo potency. Corresponding studies of cross-binding to the mitogenic IGF receptor (Assay: hIGFR) demonstrated affinities similar to native insulin.

TABLE 3

| | In Vitro Data | | | | | | |
|---|---|---|---|---|---|---|---|
| | hIR-A | | hIR-B | | IGFR | | ΔGU |
| Analogue | Kd | error | Kd | error | Kd | error | kcal/mol |
| GluA13 LysB28 ProB29 | 0.14 | 0.02 | 0.23 | 0.03 | 9.03 | 1.48 | ND |
| GluA13 parachloro- | 0.23 | 0.03 | 0.31 | 0.04 | 27.8 | 5.30 | ND |
| PheB24 LysB28 ProB29 | | | | | | | |
| HisA13 LysB28 ProB29 | 0.04 | 0.01 | 0.07 | 0.01 | 2.87 | 0.45 | ND |
| TrpA13 LysB28 ProB29 | ND | | 0.11 | 0.02 | ND | | 2.6 ± 0.1 |
| TrpA13 parachloro- | ND | | 0.07 | 0.01 | ND | | 2.5 ± 0.1 |
| PheB24 LysB28 ProB29 | | | | | | | |
| GlnAS8 TrpA13 LysB28 | 0.03 | 0.01 | 0.07 | 0.01 | 4.64 | 0.71 | ND |
| ProB29 | | | | | | | |
| GlnA8 TrpA13 parachloro- | ND | | 0.04 | 0.01 | ND | | ND |
| PheB24 LysB28 ProB29 | | | | | | | |
| TryA13 LysB28 ProB29 | ND | | 15.4 | 2.7 | ND | | 2.8 ± 0.1 |
| TyrA13 parachloro- | ND | | 0.03 | 0.01 | ND | | 2.5 ± 0.1 |
| PheB24 LysB28 ProB29 | | | | | | | |
| TyrA13 orthochloro- | ND | | 0.06 | 0.01 | ND | | 2.1 ± 0.1 |
| PheB24 LysB28 ProB29 | | | | | | | |
| TyrA13 cyclohexylalanine- | ND | | 0.22 | 0.03 | ND | | ND |
| B24 LysB28 ProB29 | | | | | | | |
| AlaA13 LysB28 ProB29 | 0.05 | 0.01 | 0.17 | 0.03 | 9.7 | 1.6 | ND |
| GluB17 LysB28 ProB29 | 0.06 | 0.01 | 0.11 | 0.02 | 2.64 | 0.42 | ND |
| PheA13 LysB28 ProB29 | 0.05 | 0.01 | 0.07 | 0.01 | 3.12 | 0.51 | ND |
| PheB17 LysB28 ProB29 | 0.07 | 0.01 | 0.07 | 0.01 | 3.03 | 0.48 | ND |

ND: Not done

To evaluate the biological activity (potency and duration of action) of the analogues in an animal model, male Sprague-Dawley rats were rendered diabetic by treatment with streptozotocin (STZ). The activity of the insulin analogues was evaluated in relation to that of Humalog® (U-100 strength taken from an unexpired commercial vial). 5, 20 or 60 micrograms of each of the analogue formulations were injected subcutaneously, and resulting changes in blood glucose concentration were monitored by serial measurements using a clinical glucometer (Hypoguard Advance Micro-Draw meter). Rats were injected subcutaneously at time t=0 in groups of five (N=4-6). Blood was obtained from the clipped tip of the tail at time 0 and every 10 minutes up to 360 min to determine the drop in blood glucose as Δ/min and Δ/hr over the 1st hour. Representative analogues of the present invention were found, under conditions of formulation similar to that of Humalog®, to retain a substantial proportion of the biological activity of insulin and with duration of action foreshortened with respect to Humalog®.

TABLE 4

| Analogues (substitutions as | Small Animal Activity (rat) | | |
|---|---|---|---|
| indicated, all other positions | | Δ Blood Glucose | |
| are wild-type human insulin) | | (60 ug dose) | error |

| | | | |
|---|---|---|---|
| GluA13 LysB28 ProB29 | −303.34 | Δ/hr | 8.86 |
| | −5.16 | Δ/min | 0.15 |
| GluA13 parachloro-PheB24 | −310.54 | Δ/hr | 8.5 |
| LysB28 ProB29 | −5.18 | Δ/min | 0.14 |
| TrpA13 LysB28 ProB29 | −256.03* | Δ/hr | 17.91 |
| | −4.26* | Δ/min | 0.3 |
| TrpA13 parachloro-PheB24 | −218.89 | Δ/hr | 35.61 |
| LysB28 ProB29 | −3.65 | Δ/min | 0.61 |
| GlnA8 TrpA13 LysB28 ProB29 | −206.36 | Δ/hr | 29.8 |
| | −3.44 | Δ/min | 0.5 |
| GlnA8 TrpA13 parachloro- | −287.96 | Δ/hr | 24.87 |
| PheB24 LysB28 ProB29 | −4.80 | Δ/min | 0.41 |
| TryA13 LysB28 ProB29 | −300.38 | Δ/hr | 28.53 |
| | −5.01 | Δ/min | 0.48 |
| TyrA13 parachloro-PheB24 | −239.66 | Δ/hr | 20.21 |
| LysB28 ProB29 | −3.99 | Δ/min | 0.34 |
| TyrA13 orthochloro-PheB24 | −303.75 | Δ/hr | 23.82 |
| LysB28 ProB29 | −5.06 | Δ/min | 0.4 |
| TyrA13 cyclohexylalanine- | −263.84 | Δ/hr | 5.92 |
| B24 LysB28 ProB29 | −4.40 | Δ/min | 0.1 |
| AlaA13 LysB28 ProB29 | −272.06 | Δ/hr | 19.53 |
| | −4.93 | Δ/min | 0.33 |
| GluB17 LysB28 ProB29 | −296.83 | Δ/hr | 14.87 |
| | −4.95 | Δ/min | 0.25 |
| PheA13 LysB28 ProB29 | −300.26 | Δ/hr | 33 |
| | −5.00 | Δ/min | 0.55 |
| PheB17 LysB28 ProB29 | −259.14 | Δ/hr | 6.22 |
| | −4.32 | Δ/min | 0.1 |

Non-diabetic anesthetized Sinclair pigs whose pancreatic β- and α-cell function has been suppressed by IV octreotide acetate were used to assess large animal in vivo effects and pharmacodynamics. Approximately 30 minutes after initiating octreotide acetate infusion, baseline euglycemia was established with 10% dextrose infusion. Once in a euglycemic state, 0.1-0.2 U/kg insulin was administered intravenously through a vascular access port at. In order to quantify peripheral insulin-mediated glucose uptake, blood glucose was measured every 5 minutes while a variable rate glucose infusion maintained a blood glucose level of approximately 85 mg/dL. This glucose infusion was maintained until the endogenous blood glucose returned to baseline (pre-insulin infusion) levels. Pharmacodynamic (PD) effects were measured as time to half-maximal effect (T_{1/2} early), time to half-maximal effect (T_{1/2} late), and time to maximal effect (Tₘₐₓ). For each of these analyses, the 20-minute moving mean curve fit was employed. Representative analogues of the present invention demonstrated large animal biological effects comparable to native insulin as shown in Table 5.

TABLE 5

| Analogues (substitutions as | |
|---|---|
| indicated, all other positions | Large Animal |
| are wild-type human insulin) | Activity (pig) |
| | 0.1-0.2 Ukg dose |
| TrpA13 parachloro-PheB24 LysB28 ProB29 | 10.0 min T½ early |
| | 56.0 min T½ late |
| TyrA13 parachloro-PheB24 LysB28 ProB29 | 12.0 min T½ early |
| | 54.0 min T½ late |
| TyrA13 cyclohexylalanine-B24 | 8.0 min T½ early |
| LysB28 ProB29 | |
| | 58.0 min T½ late |

A method for treating a patient with diabetes mellitus comprises administering a single-chain insulin analogue as described herein. It is another aspect of the present invention that the single-chain insulin analogues may be prepared either in yeast (Pichia pastoris) or subject to total chemical synthesis by native fragment ligation. We further envision the analogues of the present invention providing a method for the treatment of diabetes mellitus or the metabolic syndrome. The route of delivery of the insulin analogue is by subcutaneous injection through the use of a syringe or pen device.

A single-chain insulin analogue of the present invention may also contain other modifications, such as a halogen atom at positions B24, B25, or B26 as described more fully in co-pending U.S. patent application Ser. No. 13/018,011, the disclosure of which is incorporated by reference herein. An insulin analogue of the present invention may also contain a foreshortened B-chain due to deletion of residues B1-B3 as described more fully in co-pending U.S. Provisional Patent Application 61/589,012.

A pharmaceutical composition may comprise such insulin analogues and which may optionally include zinc. Zinc ions may be included at varying zinc ion:protein ratios, ranging from 2.2 zinc atoms per insulin analogue hexamer to 3 zinc atoms per insulin analogue hexamer. The pH of the formulation is in the range pH 6.8-8.0. In such a formulation, the concentration of the insulin analogue would typically be between about 0.6-5.0 mM; concentrations up to 5 mM may be used in vial or pen; the more concentrated formulations (U-200 or higher) may be of particular benefit in patients with marked insulin resistance. Excipients may include glycerol, glycine, arginine, Tris, other buffers and salts, and anti-microbial preservatives such as phenol and meta-cresol; the latter preservatives are known to enhance the stability of the insulin hexamer. Single-chain insulin analogues may be formulated in the presence of zinc ions or in their absence. Such a pharmaceutical composition as described above may be used to treat a patient having diabetes mellitus or other medical condition by administering a physiologically effective amount of the composition to the patient.

Based upon the foregoing disclosure, it should now be apparent that the two-chain or single-chain insulin analogues provided will carry out the objects set forth hereinabove. Namely, these insulin analogues exhibit both accelerated absorption into the blood stream from a subcutaneous depot (“fast on”) and foreshortened duration of signaling (“fast off”) while maintaining at least a fraction of the biological activity of wild-type insulin. It is, therefore, to be understood that any variations evident fall within the scope of the claimed invention and thus, the selection of specific component elements can be determined without departing from the spirit of the invention herein disclosed and described.

The following literature is cited to demonstrate that the testing and assay methods described herein would be understood by one of ordinary skill in the art.
Glendorf, T., Knudsen, L., Stidsen, C. E., Hansen, B. F., Hegelund, A. C., Sørensen, A. R., Nishimura, E., & Kjeldsen, T. 2012. Systematic evaluation of the metabolic to mitogenic potency ratio for B 10-substituted insulin analogues. PLoS One 7(2), e29198.
Hohsaka, T., & Sisido, M. 2012. Incorporation of non-natural amino acids into proteins. Curr. Opin. Chem. Biol. 6, 809-15.
Hua, Q. X., Nakagawa, S. H., Jia, W., Huang, K., Phillips, N. B., Hu, S. & Weiss, M. A. (2008) Design of an active ultrastable single-chain insulin analog: synthesis, structure, and therapeutic implications. J. Biol. Chem. 283, 14703-14716.
Kristensen, C., Andersen, A. S., Hach, M., Wiberg, F. C., Schäffer, L., & Kjeldsen, T. 1995. A single-chain insulin-like growth factor I/insulin hybrid binds with high affinity to the insulin receptor. Biochem. J. 305, 981-6.
Lee, H. C., Kim, S. J., Kim, K. S., Shin, H. C., & Yoon, J. W. 2000. Remission in models of type 1 diabetes by gene therapy using a single-chain insulin analogue. Nature 408, 483-8. Retraction in: Lee H C, Kim K S, Shin H C. 2009. Nature 458, 600.
Phillips, N. B., Whittaker, J., Ismail-Beigi, F., & Weiss, M. A. (2012) Insulin fibrillation and protein design: topological resistance of single-chain analogues to thermal degradation with application to a pump reservoir. J. Diabetes Sci. Technol. 6, 277-288.
Sciacca, L., Cassarino, M. F., Genua, M., Pandini, G., Le Moli, R., Squatrito, S., & Vigneri, R. 2010. Insulin analogues differently activate insulin receptor isoforms and post-receptor signaling. Diabetologia 53, 1743-53.
Wang, Z. X. 1995. An exact mathematical expression for describing competitive biding of two different ligands to a protein molecule FEBS Lett. 360: 111-114.
Whittaker, J., and Whittaker, L. 2005. Characterization of the functional insulin binding epitopes of the full-length insulin receptor. J. Biol. Chem. 280: 20932-20936.

(human proinsulin)
SEQ ID NO: 1
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Ala-Glu-Asp-
Leu-Gln-Val-Gly-Gln-Val-Glu-Leu-Gly-Gly-Gly-Pro-
Gly-Ala-Gly-Ser-Leu-Gln-Pro-Leu-Ala-Leu-Glu-Gly-
Ser-Leu-Gln-Lys-Arg-Gly-Ile-Val-Glu-Gln-Cys-Cys-
Thr-Ser-Ile-Cys-Ser-Leu-Tyr-Gln-Leu-Glu-Asn-Tyr-
Cys-Asn
(human A chain)
SEQ ID NO: 2
Gly-Ile-Val-Glu-Gln-Cys-Cys-Thr-Ser-Ile-Cys-Ser-
Leu-Tyr-Gln-Leu-Glu-Asn-Tyr-Cys-Asn
(human B chain)
SEQ ID NO: 3
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr
(variant human A chain)
SEQ ID NO: 4
Gly-Ile-Val-Glu-Gln-Cys-Cys-Thr-Ser-Ile-Cys-Xaa-
Leu-Tyr-Gln-Leu-Glu-Asn-Tyr-Cys-Asn

Where Xaa indicates Ala, Thr, Asp, Asn Glu, Gln, His or Tyr.

(variant human A chain)
SEQ ID NO: 5
Gly-Ile-Val-Glu-Gln-Cys-Cys-Thr-Ser-Ile-Cys-
Ser-Xaa-Tyr-Gln-Leu-Glu-Asn-Tyr-Cys-Asn

Where Xaa indicates Ala, Glu, Gln, His, or Trp.

(variant human A chain)
SEQ ID NO: 6
Gly-Ile-Val-Glu-Gln-Cys-Cys-Thr-Ser-Ile-Cys-Ser-
Leu-Tyr-Gln-Leu-Xaa-Asn-Tyr-Cys-Asn

Where Xaa indicates Ala, Gln, His, Trp, or Tyr.

(variant human A chain)
SEQ ID NO: 7
Gly-Ile-Val-Glu-Gln-Cys-Cys-Thr-Ser-Ile-Cys-Xaa₁-
Xaa₂-Tyr-Gln-Leu-Xaa₃-Asn-Tyr-Cys-Asn

Where at least one of the Xaa sites contains a substitution relative to wild-type human insulin and wherein Xaa₁ indicates Ala, Thr, Asp, Asn Glu, Gln, His or Tyr; where Xaa₂ indicates Ala, Glu, Gln, His, or Trp; and where Xaa₃ indicates Ala, Gln, His, Trp, or Tyr.

(variant human A chain)
SEQ ID NO: 8
Gly-Ile-Val-Glu-Gln-Cys-Cys-Xaa₁-Ser-Ile-Cys-Xaa₂-
Xaa₃-Tyr-Gln-Leu-Xaa4-Asn-Tyr-Cys-Asn

Where at least one of the Site-2-related sites (A12, A13, and A17) contains a substitution relative to wild-type human insulin and wherein Xaa₂ indicates Ala, Thr, Asp, Asn Glu, Gln, His or Tyr; where Xaa₃ indicates Ala, Glu, Gln, His, or Trp; and where Xaa₄ indicates Ala, Gln, His, Trp, or Tyr; and where Xaa₁ indicates His, Glu, Gln, Arg, or Lys.

(variant human B chain)
SEQ ID NO: 9
Phe-Val-Asn-Gln-His-Leu-Cys-Gly-Ser-His-Leu-Val-
Xaa₃-Ala-Leu-Tyr-Leu-Val-Cys-Gly-Glu-Arg-Gly-
Phe-Phe-Tyr-Thr-Xaa₁-Xaa₂-Thr

Where Xaa₃ indicates Ala, Asp, His, or Leu; where Xaa₁ indicates any amino acid excluding Proline, Glycine, Tryptophan, Phenylalanine, Tyrosine, and Cysteine; and where Xaa₂ indicates Pro, Glu or Lys.

(variant human B chain)
SEQ ID NO: 10
Phe-Val-Asn-Gln-His-Leu-Cys-Gly-Ser-His-Leu-Val-
Glu-Ala-Leu-Tyr-Xaa₃-Val-Cys-Gly-Glu-Arg-Gly-Phe-
Phe-Tyr-Thr-Xaa₁-Xaa₂-Thr

Where Xaa₃ indicates Glu, Gln, Ala, His, Trp, or Tyr; where Xaa₁ indicates any amino acid excluding Proline, Glycine, Tryptophan, Phenylalanine, Tyrosine, and Cysteine; and where Xaa₂ indictes Pro, Glu, or Lys.

(variant human B chain)
SEQ ID NO: 11
Phe-Val-Asn-Gln-His-Leu-Cys-Gly-Ser-His-Leu-Val-
Xaa₁-Ala-Leu-Tyr-Xaa₂-Val-Cys-Gly-Glu-Arg-Gly-
Phe-Phe-Tyr-Thr-Xaa₃-Xaa₄-Thr

Where Xaa₁ indicates Ala, Asp, His, or Leu; where Xaa₂ indicates Gln, Glu, Ala, His, Trp, or Tyr; where Xaa₃ indicates any amino acid excluding Proline, Glycine, Tryptophan, Phenylalanine, Tyrosine, and Cysteine; and where Xaa₄ indictes Pro, Glu, or Lys.

SEQ ID NO: 12
Phe-Val-Asn-Gln-His-Leu-Cys-Gly-Ser-Xaa₁-Leu-Val-
Xaa₁-Ala-Leu-Tyr-Xaa₂-Val-Cys-Gly-Glu-Arg-Gly-Phe-
Phe-Tyr-Thr-Xaa₆-Xaa₇-Thr-Gly-Z-Arg-Arg-Gly-Ile-
Val-Glu-Gln-Cys-Cys-Xaa₈-Ser-Ile-Cys-Xaa₃-Xaa₄-
Xaa₉-Gln-Leu-Xaa₅-Asn-Tyr-Cys-Asn

Where at least one of the Site-2-related sites (B13, B17, A12, A13, and A17) contains a substitution relative to wild-type human insulin wherein Xaa₁ indicates Glu, Ala, Asp, His, or Leu; where Xaa₂ indicates Leu, Glu, Gln, Ala, His, Trp, or Tyr; where Xaa₃ indicates Ser, Ala, Thr, Asp, Asn, Glu, Gln, or His; where Xaa₄ indicates Leu, Ala, Glu, Gln, His, or Trp; where Xaa₅ indicates Glu, Gln, Ala, His, or Leu; where Xaa₁ indictes His or Asp; where Xaa₆ indicates any amino acid excluding Proline, Glycine, Tryptophan, Phenylalanine, Tyrosine, and Cysteine; where Xaa₇ indictes Pro or Lys; Xaa₈ indictes Glu, Gln, His, Arg, Lys or Ornithine; where Xaa₉ indicates Tyr or Glu; and where Z indicates a polypeptide segment of length 3-8.

SEQ ID NO: 13
Phe-Val-Asn-Gln-His-Leu-Cys-Gly-Ser-Xaa₁-Leu-Val-
Xaa₁-Ala-Leu-Tyr-Xaa₂-Val-Cys-Gly-Glu-Arg-Gly-
Phe-Phe-Tyr-Thr-Xaa₆-Xaa₇-Thr-Glu-Glu-Z-Arg-
Arg-Gly-Ile-Val-Glu-Gln-Cys-Cys-Xaa₈-Ser-Ile-
Cys-Xaa₃-Xaa₄₋Xaa₉-Gln-Leu-Xaa₅-Asn-Tyr-Cys-Asn

Where at least one of the Site-2-related sites (B13, B17, A12, A13, and A17) contains a substitution relative to wild-type human insulin wherein Xaa₁ indicates Glu, Ala, Asp, His, or Leu; where Xaa₂ indicates Leu, Glu, Gln, Ala, or His; where Xaa₃ indicates Ser, Ala, Thr, Asp, Asn, Glu, Gln, or His; where Xaa₄ indicates Leu, Ala, Glu, Gln, His, or Trp; where Xaa₅ indicates Glu, Gln, Ala, His, or Leu; where Xaa₁ indictes His or Asp; where Xaa₆ indicates any amino acid excluding Proline, Glycine, Tryptophan, Phenylalanine, Tyrosine, and Cysteine; where Xaa₇ indictes Pro or Lys; Xaa₈ indictes Glu, Gln, His, Arg, Lys or Ornithine; where Xaa₉ indicates Tyr or Glu; and where Z indicates a polypeptide segment of length 2-7.

SEQ ID NO: 14
Phe-Val-Asn-Gln-His-Leu-Cys-Gly-Ser-Xaa₁-Leu-Val-
Xaa₁-Ala-Leu-Tyr-Xaa₂-Val-Cys-Gly-Glu-Arg-Gly-
Phe-Phe-Tyr-Thr-Xaa₆-Xaa₇-Thr-Glu--Z-Ser-Arg-
Gly-Ile-Val-Glu-Gln-Cys-Cys-Xaa₈-Ser-Ile-Cys-
Xaa₃-Xaa₄-Xaa₉-Gln-Leu-Xaa₅-Asn-Tyr-Cys-Asn

Where at least one of the Site-2-related sites (B13, B17, A12, A13, and A17) contains a substitution relative to wild-type human insulin wherein Xaa₁ indicates Glu, Ala, Asp, His, or Leu; where Xaa₂ indicates Leu, Glu, Gln, Ala, or His; where Xaa₃ indicates Ser, Ala, Thr, Asp, Asn, Glu, Gln, or His; where Xaa₄ indicates Leu, Ala, Glu, Gln, His, or Trp; where Xaa₅ indicates Glu, Gln, Ala, His, or Leu; where Xaa₁ indictes His or Asp; where Xaa₆ indicates any amino acid excluding Proline, Glycine, Tryptophan, Phenylalanine, Tyrosine, and Cysteine; where Xaa₇ indictes Pro or Lys; Xaa₈ indictes Glu, Gln, His, Arg, Lys or Ornithine; where Xaa₉ indicates Tyr or Glu; and where Z indicates a polypeptide segment of length 3-8.

(SEQ ID NO: 15)
GIVEQCCTSICSEYQLENYCN
(SEQ ID NO: 16)
FVNQHLCGSHLVEALYLVCGERGFFYTKPT
(SEQ ID NO: 17)
FVNQHLCGSHLVEALYLVCGERGXFYTKPT, X =
parachlorophenylalanine
(SEQ ID NO: 18)
GIVEQCCTSICSHYQLENYCN
(SEQ ID NO: 19)
GIVEQCCTSICSHYQLENYCN
(SEQ ID NO: 20)
GIVEQCCTSICSWYQLENYCN
(SEQ ID NO: 21)
FVNQHLCGSDLVEALYLVCGERGXFYTKPT, X =
orthofluorophenylalanine
(SEQ ID NO: 22)
GIVEQCCQSICSWYQLENYCN
(SEQ ID NO: 23)
GIVEQCCTSICSYYQLENYCN
(SEQ ID NO: 24)
FVNQHLCGSHLVEALYLVCGERGXFYTKPT, X =
orthochlorophenylalanine
(SEQ ID NO: 25)
FVNQHLCGSHLVEALYLVCGERGXFYTKPT, X =
cyclohexylalanine
(SEQ ID NO: 26)
GIVEQCCTSICSAYQLENYCN
(SEQ ID NO: 27)
FVNQHLCGSHLVEALYEVCGERGFFYTKPT
(SEQ ID NO: 28)
GIVEQCCTSICSFYQLENYCN
(SEQ ID NO: 29)
GIVEQCCTSICSLYQLENYCN
(SEQ ID NO: 30)
GIVEQCCQSICSWYQLENYCN
(SEQ ID NO: 31)
FVNQHLCGSHLVQALYLVCGERGXFYTKPT, X =
parachlorophenylalanine

What is claimed is:

1. An insulin analogue containing at least one substitution relative to wild type insulin comprising a substitution at a position selected from the group consisting of:
an Ala, Asp, His, or Leu substitution at B13,
a Glu, Gln, Ala, His, Trp or Tyr substitution at B17,
an Ala, Thr, Asp, Asn, Glu, Gln, His or Tyr substitution at A12,
an Ala, Glu, Gln, His, Tyr, Phe or Trp substitution at A13, and
an Ala, Gln, His, Trp, or Tyr substitution at A17.

2. An insulin analogue of claim 1 additionally comprising an Asp or Lys substitution at position B28.

3. An insulin analogue of claim 2 additionally comprising a Pro substitution at position B29.

4. An insulin analogue of claim 1, wherein the analogue contains a Glu, His, Trp, Tyr, Ala or Phe substitution at position A13.

5. An insulin analogue according to claim 4, additionally comprising a substitution at position B24 selected from the group consisting of para-chloro Phenylalanine, ortho-fluoro Phenylalanine and cyclohexanylalanine.

6. The insulin analogue of claim 5 comprising an ortho-fluoro-Phenylalanine substitution at position B24 and additionally comprising an Aspartic Acid substitution at position B10.

7. The insulin analogue of claim 1, wherein the analogue contains an Alanine substitution at position B17.

8. The insulin analogue of claim 1, wherein the analogue contains an Alanine or Phenylalanine substitution at position A13.

9. The insulin analogue of claim 1, wherein the analogue contains an Glu substitution at position A17.

10. The insulin analogue of claim 1, wherein the A-chain sequence is selected from SEQ ID NOS: 4-8.

11. The insulin analogue of claim 1, wherein the B-chain sequence is selected from SEQ ID NOS: 9-11.

12. A nucleic acid encoding an A chain of an insulin analogue containing at least one substitution relative to wild type insulin, wherein the substitution is selected from the group consisting of:
an Ala, Thr, Asp, Asn, Glu, Gln, His or Tyr substitution at A12,
an Ala, Glu, Gln, His, Tyr, Phe or Trp substitution at A13, and
an Ala, Gln, His, Trp, or Tyr substitution at A17.

13. A nucleic acid according to claim 12 encoding an A chain polypeptide of an insulin analogue comprising SEQ ID NO: 4, 5, 6, 7, or 8.

14. A nucleic acid encoding a B chain of an insulin analogue containing at least one substitution relative to wild type insulin, wherein the substitution is selected from the group consisting of:
an Ala, Asp, His, or Leu substitution at B13, and
a Glu, Gln, Ala, His, Trp or Tyr substitution at B17.

15. A nucleic acid according to claim 14 encoding a B chain polypeptide of an insulin analogue comprising SEQ ID NO: 9, 10, or 11.

16. A nucleic acid according to claim 14 additionally comprising a nonsense codon at position B24.

17. A method of lowering the blood sugar of a patient, the method comprising administering a physiologically effective amount of an insulin analogue or a pharmaceutically acceptable salt thereof to the patient, wherein the insulin analogue comprises at least one substitution relative to wild type insulin selected from the group consisting of:
an Ala, Asp, His, or Leu substitution at B13,
a Glu, Gln, Ala, His, Trp or Tyr substitution at B17,
an Ala, Thr, Asp, Asn, Glu, Gln, His or Tyr substitution at A12,
an Ala, Glu, Gln, His, Tyr, Phe or Trp substitution at A13, and
an Ala, Gln, His, Trp, or Tyr substitution at A17 .

18. The method of claim 17, wherein the analogue is formulated in a composition containing zinc ions at a molar ratio of between 2 and 10 zinc ions per six single-chain insulin analogue monomers and wherein the pH of the formulation is between pH 6.8 and pH 8.0.

19. The method of claim 18, wherein the insulin analogue is formulated at a strength of at least U-100.

20. The method of claim 19, wherein the insulin analogue is formulated at a strength of between U-500 and U-1000.
